(19) United States
(12) Patent Application Publication
(10) Pub. No.: US 2025/0207189 A1
(43) Pub. Date: Jun. 26, 2025

(54) DINUCLEOTIDE STOCHASTIC SEQUENCING

(71) Applicant: 10x Genomics, Inc., Pleasanton, CA (US)

(72) Inventors: Anton Jan BJÖRNINEN, Stockholm (SE); Debashish CHITNIS, Stockholm (SE); Toon VERHEYEN, Solna (SE)

(21) Appl. No.: 18/991,062

(22) Filed: Dec. 20, 2024

Related U.S. Application Data
(60) Provisional application No. 63/614,355, filed on Dec. 22, 2023.

(51) Int. Cl. C12Q 1/6869; C12Q 1/6844

(52) U.S. Cl. CPC C12Q 1/6869; C12Q 1/6844

(57) ABSTRACT

The present disclosure relates in some aspects to methods, systems, and kits for sequencing a template nucleic acid molecule, where the methods comprise: (i) contacting a priming strand bound to the template nucleic acid molecule with a polymerase and a plurality of dinucleotide molecules to form a complex comprising the 3′ terminus of the priming strand, the template nucleic acid molecule, the polymerase, and a dinucleotide molecule, where the priming strand comprises a reversibly terminated nucleotide at its 3′ end such that the dinucleotide molecule in the complex is not incorporated into the priming strand; and ii) detecting a presence of the dinucleotide molecule in the complex to identify a complementary nucleotide in the template nucleic acid molecule.

### CROSS-REFERENCE TO RELATED APPLICATIONS

This application claims the priority benefit of U.S. Provisional Patent Application Ser. No. 63/614,355, filed Dec. 22, 2023, the contents of which are incorporated herein by reference in their entirety.

### FIELD

The present disclosure relates in some aspects to methods for sequencing nucleic acid molecules, including methods for in situ sequencing and analysis of target nucleic acids in a biological sample.

### BACKGROUND

The conventional sequencing-by-synthesis (SBS) method for sequencing nucleic acid molecules is based on incorporation of a fluorescent, reversibly terminated nucleotide into an extended priming strand, where the incorporated nucleotide is complementary to a nucleotide at the position of the template nucleic acid molecule that is being probed. Both the reversible terminator and the fluorescent moiety must be cleaved off the newly incorporated nucleotide before progressing to the next cycle of sequencing, leaving a “molecular scar” that destabilizes the extended priming strand and limits the length of template sequence that can be sequenced. Thus, improved methods for sequencing nucleic acid molecules that simplify the steps required, improve signal detection and/or that minimize the “molecular scarring” phenomenon are of interest in the field.

Recently, new sequencing technologies have emerged that attempt to improve signal detection while circumventing the “molecular scarring” phenomenon by incorporating a reversible terminator at the 3′ terminus of the priming strand and performing stochastic binding of a polymerase and a fluorescent nucleotide to form a bound complex. As the fluorescent nucleotide is not incorporated into the priming strand (but can still be detected as part of the bound complex), it does not leave a molecular scar and thus enables the ability to sequence longer template sequences in a single sequence read. As the formation of the bound complex is driven by stochastic binding, complex formation is primarily driven by local concentrations of the polymerase and nucleotides, thereby leading to an inherent instability of the complex upon rinsing away unbound polymerase and fluorescent nucleotides. Such stochastic binding-based methods are promising; however, there is a need in the field for methods that increase stability of the transiently formed complex.

### SUMMARY

Disclosed herein are methods for sequencing nucleic acid molecules that include contacting a priming strand that includes a 3′ reversibly terminated nucleotide and is bound to a template nucleic acid molecule with a polymerase and a dinucleotide molecule comprising a first (5′) nucleotide moiety and a second (3′) nucleotide moiety, where the dinucleotide molecule is not incorporated into the priming strand but the complementarity of the dinucleotide molecule with the template nucleic acid enhances the stability of a complex comprising the 3′ terminus of the priming strand, the template nucleic acid molecule, the polymerase, and the complementary nucleotide to be sequenced. A detectable signal associated with the dinucleotide molecule (e.g., a fluorescent label conjugated to the second (3′) nucleotide moiety of the dinucleotide molecule, or absence of fluorescence in cases where the dinucleotide molecule is not labeled with any fluorescent label) allows detection of the presence of a complementary dinucleotide molecule in the complex. The present disclosure, in some aspects, provides methods, compositions, kits, and systems for performing dinucleotide sequencing of a template nucleic acid molecule, where mixtures of non-incorporated, detectably labeled dinucleotide molecules are used to probe the template nucleic acid sequence and create stable retention of an optical signal (e.g., a fluorescence signal or an absence thereof) associated with the presence of a complementary dinucleotide molecule in the aforementioned complex to thereby improve signal detection, minimize “molecular scarring” and enable longer sequence reads.

Also disclosed herein are methods for sequencing a template nucleic acid molecule, the methods including: contacting a priming strand bound to the template nucleic acid molecule with a polymerase and a first plurality of dinucleotide molecules to form a complex. In some embodiments, the complex includes a 3′ terminus of the priming strand, the template nucleic acid molecule, the polymerase, and a first dinucleotide molecule of the first plurality of dinucleotide molecules. In some embodiments, the priming strand includes a reversibly terminated nucleotide at its 3′ end such that the first dinucleotide molecule of the complex is not incorporated into the priming strand. In some embodiments, the method further includes detecting a presence of the first dinucleotide molecule in the complex to identify a complementary nucleotide in the template nucleic acid molecule.

Also disclosed herein are methods for sequencing a template nucleic acid molecule, the methods including providing: a priming strand bound to the template nucleic acid molecule, wherein the priming strand comprises a reversibly terminated nucleotide at its 3′ end; and one or more reagents comprising a polymerase molecule and a first plurality of dinucleotide molecules; and the methods further including contacting the priming strand bound to the template nucleic acid molecule with the one or more reagents to form a complex comprising a 3′ terminus of the priming strand, the template nucleic acid molecule, the polymerase, and a first dinucleotide molecule of the first plurality of dinucleotide molecules, wherein the first dinucleotide molecule is blocked from being incorporated into the priming strand; and detecting a presence of the first dinucleotide molecule in the complex to identify a complementary nucleotide in the template nucleic acid molecule. In some embodiments, the methods further includes deprotecting the reversibly terminated nucleotide at the 3′ end of the priming strand; and performing an extension reaction to incorporate a reversibly terminated nucleotide molecule into an extended priming strand.

In some embodiments, the first plurality of dinucleotide molecules comprises at least one of four sets of dinucleotide molecules, wherein each of the four sets comprises dinucleotide sequences comprising: a same 5′ nucleotide that is unique for the set and a 3′ nucleotide that can be the same or different among the four sets. In some embodiments, the first plurality of dinucleotide molecules comprises at least one of four sets of dinucleotide molecules, wherein each of the four sets comprises dinucleotide sequences comprising: a same 5′ nucleotide that is unique for the set and one or more different 3′ nucleotides, wherein the one or more different 3′ nucleotides across the set are capable of base pairing with A, C, and G, and at least one of T and U.

In some embodiments, the first plurality of dinucleotide molecules comprises at least one of four sets of dinucleotide molecules, wherein each of the four sets comprises four different dinucleotide sequences include a same 5′ nucleotide that is unique for the set and four different 3′ nucleotides, wherein each dinucleotide molecule of the set is associated with a same detectable signal.

In some embodiments, the first plurality of dinucleotide molecules comprises at least one of four sets of dinucleotide molecules, wherein each of the four sets comprises dinucleotide sequences comprising: a 5′ nucleotide that can be the same or different among the four sets and a same 3′ nucleotide that is unique for the set. In some embodiments, the first plurality of dinucleotide molecules comprises at least one of four sets of dinucleotide molecules, wherein each of the four sets comprises dinucleotide sequences comprising one or more different 5′ nucleotides and a same 3′ nucleotide that is unique for the set, wherein the one or more different 5′ nucleotides across the set are capable of base pairing with A, C, and G, and at least one T and U.

In some embodiments, the first plurality of dinucleotide molecules comprises at least one of four sets of dinucleotide molecules, wherein each of the four sets comprises four different dinucleotide sequences comprising: four different 5′ nucleotides and a same 3′ nucleotide that is unique for the set wherein each dinucleotide molecule of the set is associated with a same detectable signal.

In some embodiments, the methods include repeating steps (a)-(d) for at least one additional cycle using at least one additional plurality of dinucleotide molecules to detect a presence of a dinucleotide molecule in a complex and identify at least one additional complementary nucleotide in the template nucleic acid molecule.

In some embodiments, the first plurality and the at least one additional plurality of dinucleotide molecules each include at least one of four sets of dinucleotide molecules, wherein each of the four sets include four different dinucleotide sequences, the four different sequences including a same 5′ nucleotide that is unique for the set and one or more different 3′ nucleotides, wherein the one or more different 3′ nucleotides across the set are capable of base pairing with A, C, and G, and at least one of T and U. In some embodiments, the one or more different 3′ nucleotides comprises four different 3′ nucleotides. In some embodiments, each dinucleotide molecule of the set is associated with a same detectable signal.

In some embodiments, for a first set the same 5′ nucleotide is A, for a second set the same 5′ nucleotide is T or U, for a third set the same 5′ nucleotide is C, and for a fourth set the same 5′ nucleotide is G. In some embodiments, the first plurality and the at least one additional plurality of dinucleotide molecules each comprise dinucleotide sequences of all possible pairwise combinations of A, C, G, and either T or U.

In some embodiments, for the first set the same 5′ nucleotide is A, for the second set the same 5′ nucleotide is T, for the third set the same 5′ nucleotide is C, and for the fourth set the same 5′ nucleotide is G, such that the first plurality and the at least one additional plurality of dinucleotide molecules each comprise dinucleotide sequencing including all possible pairwise combinations of A, T, C, and G.

In some embodiments, the first plurality of dinucleotide molecules and the at least one additional plurality of dinucleotide molecules each comprise at least one of four sets of dinucleotide molecules, wherein each of the four sets comprises four different dinucleotide sequences comprising: one or more different 5′ nucleotides and a same 3′ nucleotide that is unique for the set, wherein the one or more different 5′ nucleotides across the set are capable of base pairing with A, C, and G, and at least one T and U. In some embodiments, the one or more different 5′ nucleotides comprise four different 5′ nucleotides. In some embodiments, each dinucleotide molecule of the set is associated with a same detectable signal.

In some embodiments, for a first set the same 3′ nucleotide is A, for a second set the same 3′ nucleotide is T or U, for a third set the same 3′ nucleotide is C, and for a fourth set the same 3′ nucleotide is G, such that the first plurality and the at least one additional plurality of dinucleotide molecules each comprise dinucleotide sequences of all possible pairwise combinations of A, C, G, and either T or U.

In some embodiments, for the first set, the same 3′ nucleotide is A, for the second set the same 3′ nucleotide is T, for the third set the same 3′ nucleotide is C, and for the fourth set the same 3′ nucleotide is G, such that the first plurality and the at least one additional plurality of dinucleotide molecules each comprise dinucleotide sequences of all possible pairwise combinations of A, T, C, and G.

In some embodiments, for at least one of the four sets, the detectable signal is associated with a detectable moiety coupled to a corresponding dinucleotide molecule. In some embodiments, for at least three of the four sets, the detectable signal is associated with a detectable moiety coupled to a corresponding dinucleotide molecule. In some embodiments, for at least one of the four sets, the detectable moiety is coupled to a 3′ nucleotide of a corresponding dinucleotide molecule. In some embodiments, the detectable moiety is coupled to a nucleobase of the 3′ nucleotide of the corresponding dinucleotide molecule. In some embodiments, the detectable moiety comprises a fluorophore. In some embodiments, the detectable signal for one of the four sets is associated with an absence of a detectable moiety. In some embodiments, the detectable signal for three of the four sets is associated with a fluorophore and the detectable signal for one of the four sets is associated with an absence of a fluorophore or other detectable moiety. In some embodiments, the detectable signal for each of the four sets comprises a detectable signal associated with a fluorophore. In some embodiments, for at least one cycle of steps (a)-(d), the dinucleotide molecule of the complex is conjugated to a detectable moiety, and detecting a presence of the dinucleotide molecule in the complex comprises detecting a signal associated with a detectable moiety. In some embodiments, for at least one cycle of steps (a)-(d), the dinucleotide molecule of the complex is not coupled to a detectable moiety, and detecting a presence of a dinucleotide molecule in the complex comprises detecting an absence of signal, and wherein the absence of signal is associated with a dinucleotide molecule that is not coupled to a detectable moiety.

In some embodiments, the at least one additional cycle includes at least 2, 3, 4, 5, 10, 20, 30, 40, or 50 additional cycles. In some embodiments, the method includes repeating steps (a)-(d) for a second cycle using a second plurality of dinucleotide molecules, to detect a presence of a dinucleotide molecule in a complex and identify a second complementary nucleotide in the template nucleic acid molecule.

In some embodiments, the method includes repeating, at least once, a pattern of: performing a first cycle of steps (a)-(d) with the first plurality of dinucleotide molecules; and performing the second cycle of steps (a)-(d) with the second plurality of dinucleotide molecules, wherein the first plurality consists of a first and a second set of the four sets of dinucleotide molecules, and the second plurality consists of a third and a fourth set of the four sets of dinucleotide molecules. In some embodiments, the detectable signal for each of the four sets is associated with a detectable moiety, and wherein the first set and the second set have different detectable moieties from each other, and the third set and the second set have different detectable moieties from each other.

In some embodiments, the method includes repeating, at least once, a pattern of: performing a first cycle of steps (a)-(d) with the first plurality of dinucleotide molecules, wherein the first plurality consists of a first set of the four sets; performing a second cycle of steps (a)-(d) with a second plurality of dinucleotide molecules, wherein the second plurality consists of a second set of the four sets; performing a third cycle of steps (a)-(d) with a third plurality of dinucleotide molecules, wherein the third plurality consists of a third set of the four sets; and performing a fourth cycle of steps (a)-(d) with a fourth plurality of dinucleotide molecules, wherein the fourth plurality consists of a fourth set of the four sets.

In some embodiments, the first plurality and the at least one additional plurality each include all four of the four sets of dinucleotide molecules, and wherein the detectable signal for each of the four sets is unique signal for that set. In some embodiments, the detectable signal for each of the four sets is a unique detectable moiety. In some embodiments, the detectable signal for three of the four sets is a unique detectable moiety, and the detectable signal for a fourth of the four sets is an absence of a detectable moiety.

In some embodiments, the first plurality of dinucleotide molecules and the at least one additional plurality of dinucleotide molecules each include sets of dinucleotide molecules that do not include a 3′ reversible terminator moiety. In some embodiments, the reversibly terminated nucleotide molecule does not include a detectable moiety. In some embodiments, the polymerase is not labeled with a detectable label.

In some embodiments, the methods further include performing a first wash step to remove unbound polymerase and unbound dinucleotide molecules prior to performing the detecting step. In some embodiments, the methods further include performing a second wash step after performing the detecting step to disrupt the complex.

In some embodiments, the methods further include, prior to performing a first contacting step, hybridizing a primer that does not comprise a reversibly terminated nucleotide at its 3′ end to a primer binding site in the template nucleic acid molecule; and performing a primer extension reaction to incorporate the 3′ reversibly terminated nucleotide into an extended primer to generate the priming strand. In some embodiments, the template nucleic acid molecule comprises a DNA molecule.

In some embodiments, In some embodiments, the template nucleic acid molecule comprises an RNA molecule. In some embodiments, the template nucleic acid molecule comprises a target analyte nucleic acid molecule. In some embodiments, the template nucleic acid molecule comprises a barcode sequence associated with a target analyte. In some embodiments, the methods further include hybridizing a circularizable probe to the target analyte or to a labeling agent bound to the target analyte and ligating the circularizable probe to form a circularized probe, wherein the method further comprises performing rolling circle amplification of the circularized probe to generate the template nucleic acid molecule. In some embodiments, the circularizable probe is a padlock probe. In some embodiments, the target analyte nucleic acid molecule comprises an mRNA molecule. In some embodiments, the template nucleic acid molecule to be sequenced is attached to a solid support. In some embodiments, the solid support comprises a sequencing flow cell. In some embodiments, the template nucleic acid molecule is sequenced in situ in a cell sample or tissue sample. In some embodiments, the template nucleic acid molecule is a nucleic acid that is endogenous to the cell sample or tissue sample. In some embodiments, the template nucleic acid molecule is a reporter nucleic acid that is a reporter for an analyte endogenous to the cell sample or tissue sample. In some embodiments, the cell or tissue sample comprises a layer of cells deposited on a surface.

In some embodiments, the complex comprises a transient complex that persists for at least 5 sec, 10 sec, 20 sec, 30 sec, 40 sec, 50 sec, 1 min, 2 min, 3 min, 4 min, 5 min, or 10 min.

Also disclosed herein are kits for sequencing a template nucleic acid molecule comprising: a primer designed to hybridize to the template nucleic acid molecule; a plurality of dinucleotide molecules; a polymerase; and reversibly terminated nucleotide molecules. In some embodiments, the primer comprises a reversibly terminated nucleotide at its 3′ end. In some embodiments, the first plurality of dinucleotide molecules comprises at least one of four sets of dinucleotide molecules, wherein each of the four sets comprises dinucleotide sequences comprising a same 5′ nucleotide that is unique for the set and one or more different 3′ nucleotides, wherein the one or more different 3′ nucleotides across the set are capable of base pairing with A, C, and G, and at least one of T and U.

In some embodiments, the plurality of dinucleotide molecules of the kit comprises four sets of four different dinucleotide sequences such that the plurality of dinucleotide molecules comprise all possible dinucleotide combinations of A, C, G, and either T or U, wherein each of the four sets comprises: a same 5′ nucleotide that is unique for the set and four different 3′ nucleotides, wherein each dinucleotide molecule of the set is associated with a same detectable signal.

In some embodiments, the first plurality of dinucleotide molecules comprises at least one of four sets of dinucleotide molecules, wherein each of the four sets comprises dinucleotide sequences comprising: one or more different 5′ nucleotides and a same 3′ nucleotide that is unique for the set, wherein the one or more different 5′ nucleotides across the set are capable of base pairing with A, C, and G, and at least one of T and U.

In some embodiments, the plurality of dinucleotide molecules of the kit comprises four sets of four different dinucleotide sequences such that the plurality of dinucleotide molecules comprise all possible dinucleotide combinations of A, C, G, and either T or U, wherein each of the four sets comprises: four different 5′ nucleotides and a same 3′ nucleotide that is unique for the set, wherein each dinucleotide molecule of the set is associated with a same detectable signal. In some embodiments, the plurality of dinucleotide molecules comprise all possible dinucleotide combinations of A, T, C, and G. In some embodiments, the plurality of dinucleotide molecules comprise all possible dinucleotide combinations of A, U, C, and G.

In some embodiments, the detectable signal for each of the sets is associated with a detectable moiety coupled to a corresponding dinucleotide molecule. In some embodiments, the detectable signal for three of the sets is associated with a detectable moiety coupled to a corresponding dinucleotide molecule, and the detectable signal for a fourth of the four sets is associated with an absence of a detectable moiety.

Also disclosed herein are systems comprising one or more processors; and a memory communicatively coupled to the one or more processors and configured to store instructions that, when executed by the one or more processors, cause the system to perform any of the methods described herein.

### BRIEF DESCRIPTION OF THE DRAWINGS

The drawings illustrate certain features and advantages of this disclosure. These embodiments are not intended to limit the scope of the appended claims in any manner.

FIG. 1 provides a non-limiting example of a process flowchart for sequencing a template nucleic acid molecule in accordance with one implementation of the methods described herein.

FIG. 2 provides a non-limiting example of a process flowchart for sequencing a template nucleic acid molecule in accordance with one implementation of the methods described herein.

FIG. 3 depicts a system for performing a sequencing assay, in accordance with some implementations of the methods described herein.

FIG. 4 depicts a computer system or computer network, in accordance with some instances of the systems described herein.

FIG. 5 provides a schematic illustration of a method for sequencing a template nucleic acid molecule in accordance with one implementation of the methods described herein.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### Overview

Methods for sequencing nucleic acid molecules are described that comprise contacting a priming strand that includes a 3′ reversibly terminated nucleotide and is bound to a template nucleic acid molecule with a polymerase and a dinucleotide molecule comprising a first (5′) nucleotide moiety and a second (3′) nucleotide moiety, where the dinucleotide molecule is not incorporated into the priming strand but the complementarity of the dinucleotide molecule with the template nucleic acid enhances the stability of a complex comprising the 3′ terminus of the priming strand, the template nucleic acid molecule, the polymerase, and the complementary dinucleotide. A detectable signal (e.g., a fluorescent label) associated with the dinucleotide molecule (e.g., a fluorophore conjugated to the second (3′) nucleotide moiety of the dinucleotide molecule) allows detection of the presence of a complementary dinucleotide molecule in the complex. The present disclosure, in some aspects, provides methods, compositions, kits, and systems for performing dinucleotide sequencing of a template nucleic acid molecule, where mixtures of non-incorporated, detectably labeled dinucleotide molecules are used to probe the template nucleic acid sequence and create stable retention of an optical signal (e.g., a fluorescence signal or absence thereof) associated with the presence of a complementary dinucleotide molecule in the aforementioned complex to thereby improve signal detection, minimize “molecular scarring” and enable longer sequence reads.

Additional aspects of the methods, compositions, kits, and systems disclosed herein are described in the sections below.

### Dinucleotide Stochastic Sequencing Methods

The dinucleotide-based sequencing methods described herein are applicable to both in situ sequencing applications (e.g., in situ sequencing of endogenous nucleic acid sequences and/or target-specific barcode sequences associated with target analytes of interest that are distributed within a cell or tissue sample) and to more conventional “sequencing in a flow cell” applications (e.g., sequencing of endogenous nucleic acid sequences extracted from a cell or tissue sample and/or sequencing of target-specific barcode sequences associated with target analytes of interest that have been prepared as described elsewhere herein and extracted from a cell or tissue sample). The in situ and flow cell sequencing approaches differ in terms of the sample preparation steps required, as described elsewhere herein, but can share common features in terms of the cyclic series of steps performed to identify nucleotides base-by-base in a template nucleic acid sequence (e.g., a target analyte sequence and/or an associated target-specific barcode sequence).

### Dinucleotide Molecules

In some instances, each cycle of a cyclic series of base-by-base sequencing reactions performed as part of the disclosed methods for in situ or flow cell sequencing may comprise contacting priming strands bound to template nucleic acid molecules with at least one dinucleotide molecule. In some instances, each cycle of a cyclic series of base-by-base sequencing reactions may comprise contacting priming strands bound to a template nucleic acid molecule with a plurality of dinucleotide molecules (e.g., a plurality of dinucleotide molecules having 2, 3, 4, 5, 6, 7, 8, 9, 10, 11, 12, 13, 14, 15, or 16 different dinucleotide sequences). Dinucleotide molecules may be synthesized using any of a variety of nucleic acid synthesis techniques known to those of skill in the art, e.g., solid-phase synthesis techniques comprising the use of phosphoramidite or phosphonate coupling chemistries (see, e.g., Roy et al. (2013), “Synthesis of DNA/RNA and Their Analogs via Phosphoramidite and H-Phosphonate Chemistries”, Molecules 18:14268-14282).

In some instances, the dinucleotide molecules used herein comprise any of a variety of naturally-occurring nucleotides and/or functional analogs thereof (e.g., nucleotide analogs capable of hybridizing to a nucleic acid sequence in a sequence-specific/correctly base-paired manner) to probe a template nucleic acid sequence. Naturally-occurring nucleotides include deoxyribonucleotides (found in DNA) that comprise a deoxyribose sugar moiety, and ribonucleotides (found in RNA) that comprise a ribose sugar moiety. Naturally-occurring deoxyribonucleotides comprise a nucleobase (or “base”) selected from the group consisting of adenine (A), thymine (T), cytosine (C), or guanine (G). Naturally-occurring ribonucleotides comprise a nucleobase selected from the group consisting of uracil (U), adenine (A), cytosine (C), or guanine (G).In some instances, the dinucleotide molecules include two deoxyribonucleotides. In some instances, the dinucleotide molecule includes a universal base. A universal base refers to a nucleotide or functional analog thereof that is capable of binding to each of A, C, G, and at least one of T and U. An example of a universal base includes inosine (I), which is a nucleoside that is capable of base-pairing with A, C, G, T, and U. Other examples of universal bases include deoxyinosine, 7-deaza-2′-deoxyinosine, 2-aza-2′-deoxyinosine, 2′-OMe inosine, 2′-F inosine, deoxy 3-nitropyrrole, 3-nitropyrrole, 2′-OMe 3-nitropyrrole, 2′-F 3-nitropyrrole, 1-(2′-deoxy-beta-D-ribofuranosyl)-3-nitropyrrole, deoxy 5-nitroindole, 5-nitroindole, 2′-OMe 5-nitroindole, 2′-F 5-nitroindole, deoxy 4-nitrobenzimidazole, 4-nitrobenzimidazole, deoxy 4-aminobenzimidazole, 4-aminobenzimidazole, deoxy nebularine, 2′-F nebularine, 2′-F 4-nitrobenzimidazole, PNA-5-introindole, PNA-nebularine, PNA-inosine, PNA-4-nitrobenzimidazole, PNA-3-nitropyrrole, morpholino-5-nitroindole, morpholino-nebularine, morpholino-inosine, morpholino-4-nitrobenzimidazole, morpholino-3-nitropyrrole, phosphoramidate-5-nitroindole, phosphoramidate-nebularine, phosphoramidate-inosine, phosphoramidate-4-nitrobenzimidazole, phosphoramidate-3-nitropyrrole, 2′-O-methoxyethyl inosine, 2′-O-methoxyethyl nebularine, 2′-O-methoxyethyl 5-nitroindole, 2′-O-methoxyethyl 4-nitro-benzimidazole, and 2′-O-methoxyethyl 3-nitropyrrole. Examples of nucleotides having universal bases include 2′-deoxy-7-azaindole-5′-triphosphate (d7AITP), 2′-deoxy-isocarbostyril-5′-triphosphate (dICSTP), 2′-deoxy-propynylisocarbostyril-5′-triphosphate (dPICSTP), 2′-deoxy-6-methyl-7-azaindole-5′-triphosphate (dM7AITP), 2′-deoxy-imidizopyridine-5′-triphosphate (dImPyTP), 2′-deoxy-pyrrollpyrizine-5′-triphosphate (dPPTP), 2′-deoxy-propynyl-7-azaindole-5′-triphosphate (dP7AITP), and 2′-deoxy-allenyl-7-azaindole-5′-triphosphate (dA7AITP). In some instances, the dinucleotide molecules include two ribonucleotides. In some instances, the dinucleotide molecules include a deoxyribonucleotide and a ribonucleotide. In some instances, the nucleotides may be conjugated to a detectable moiety, e.g., a fluorophore. In some instances, the nucleotides may be conjugated to other moieties, e.g., reactive functional groups.

In some instances, the plurality of dinucleotide molecules contacted with the primed template nucleic acid molecule(s) in each cycle of a multi-cycle sequencing process may comprise pairwise combinations of A, C, G, and T and/or U. In some instances, the set of one or more dinucleotide molecules contacted with the primed template nucleic acid molecule(s) in each cycle of a multi-cycle sequencing process may comprise pairwise combinations of A, C, G, and T.

In some instances, the plurality of dinucleotide molecules includes at least one of four sets of dinucleotide molecules, wherein the four sets each comprises: (a) a same 5′ nucleotide that is unique for the set and (b) one or more different 3′ nucleotides in the set, wherein the one or more different 3′ nucleotides across the set are capable of base-pairing with each of A, C, G, and at least one of T and U. In some embodiments, the one or more 3′ nucleotides across the set capable of base-pairing with each of A, C, G, and at least one of T and U comprises a universal base.

In some instances, the plurality of dinucleotide molecules includes at least one of four sets of dinucleotide molecules, wherein the four sets each comprises multiple (e.g., two, three, or four) different dinucleotide sequences, with the four sequences within the set including: (a) a same 5′ nucleotide that is unique for the set, and a same or different 3′ nucleotide, or (b) a same or different 5′ nucleotide, and a same 3′ nucleotide that is unique for the set, wherein molecules of each set share a same detectable signal across the set. In some embodiments, the same detectable signal shared by dinucleotide molecules of a particular set is associated with a detectable moiety such as a fluorophore conjugated to the dinucleotide molecules. In some embodiments, the same detectable signal shared by dinucleotide molecules of a particular set is an absence of signal, e.g., the dinucleotide molecules of the particular set are not coupled to a detectable moiety, or any detectable moiety coupled to the dinucleotide molecules is simply not detected or recorded.

In some embodiments, each of the at least one additional cycle comprises using four sets of dinucleotide molecules, wherein each of the four sets comprises four different dinucleotide sequences comprising: i) a same 5′ nucleotide that is unique for the set and four different 3′ nucleotides, wherein each dinucleotide molecule of the set is associated with a same detectable signal; or ii) four different 5′ nucleotides and a same 3′ nucleotide that is unique for the set, wherein each dinucleotide molecule of the set is associated with a same detectable signal; and wherein the detectable signals of the four sets are distinguishable from one another.

In some instances, the plurality of dinucleotide molecules includes at least one of four sets of dinucleotide molecules, wherein the four sets each comprises four different dinucleotide sequences, with the four sequences within the set including: (a) a same 5′ nucleotide that is unique for the set and four different 3′ nucleotides, or (b) four different 5′ nucleotides and a same 3′ nucleotide that is unique for the set, wherein molecules of each set share a same detectable signal across the set.

In some aspects, each set of the four sets includes a same 5′ nucleotide that is unique for the set and four different 3′ nucleotides. In some aspects, the four sets of dinucleotide molecules include: a first set, wherein the same 5′ nucleotide is A; a second set wherein the same 5′ nucleotide is T or U; a third set, wherein the same 5′ nucleotide is C; and a fourth set, wherein the same 5′ nucleotide is G, such that the four sets of dinucleotide molecules include dinucleotide sequences of all possible pairwise combinations of A, C, G, and either T or U. In some aspects, the four sets of dinucleotide molecules include: a first set, wherein the same 5′ nucleotide is A; a second set wherein the same 5′ nucleotide is T; a third set, wherein the same 5′ nucleotide is C; and a fourth set, wherein the same 5′ nucleotide is G, such that the four sets of dinucleotide molecules include dinucleotide sequences of all possible pairwise combinations of A, C, G, and T.

In some aspects, each set of the four sets includes four different 5′ nucleotides and a same 3′ nucleotide that is unique for the set. In some aspects, the four sets of dinucleotide molecules include: a first set, wherein the same 3′ nucleotide is A; a second set wherein the same 3′ nucleotide is T or U; a third set, wherein the same 3′ nucleotide is C; and a fourth set, wherein the same 3′ nucleotide is G, such that the four sets of dinucleotide molecules include dinucleotide sequences of all possible pairwise combinations of A, C, G, and either T or U. In some aspects, the four sets of dinucleotide molecules include: a first set, wherein the same 3′ nucleotide is A; a second set wherein the same 3′ nucleotide is T; a third set, wherein the same 3′ nucleotide is C; and a fourth set, wherein the same 3′ nucleotide is G, such that the four sets of dinucleotide molecules include dinucleotide sequences of all possible pairwise combinations of A, C, G, and T.

In some instances, the methods include use of a first plurality of dinucleotide molecules and at least one additional plurality of dinucleotide molecules. In some instances, the methods involve a multi-cycle sequencing process and the cycles include a repeating pattern of steps using multiple pluralities of dinucleotide molecules, such that across the repeating pattern of cycles all four sets of dinucleotide molecules (with sixteen different sequences) are utilized.

In some instance, the pluralities of dinucleotide molecules used in each cycle of a multi-cycle sequencing process each include the same sets of dinucleotide sequences (e.g., the pluralities of dinucleotide molecules used in each cycle may comprise the same selection of sixteen dinucleotide sequences comprising pairwise combinations of A, C, G, and T or U). In some instances, the plurality of dinucleotide molecules used in each sequencing cycle includes all four sets of dinucleotide molecules such that sixteen different dinucleotide sequences are present. In some instances, all four sets are used in a same cycle, and the detectable signal for each set is a unique detectable signal for that set. In some instances, the unique detectable signals for each of the four sets is a unique detectable moiety (e.g., a fluorophore conjugated to the molecule). In some instances, the unique detectable signal for three of the four sets is a detectable moiety (e.g., a fluorophore conjugated to the molecule), and the unique detectable signal for a fourth of the four sets is an absence of a detectable moiety.

In some instances, the pluralities of dinucleotide molecules contacted with the primed template nucleic acid molecule(s) comprise different sets of sequences in different cycles of a multi-cycle sequencing process (e.g., a first cycle utilizing a first plurality of dinucleotide molecules including two of the four sets such that eight dinucleotide sequences are present, and a second cycle utilizing a second plurality of dinucleotide molecules including the other two of the four sets such that eight different dinucleotide sequences are present).

In some aspects, the detectable signal associated with dinucleotide molecules of a set is a detectable moiety coupled to a corresponding dinucleotide molecule. Examples of detectable moieties include fluorophores, radioisotopes, molecules capable of a colorimetric reaction, magnetic particles, and any other suitable molecule or compound capable of detection. In some instances, at least one of the sets of dinucleotide molecules may comprise dinucleotide molecules that are not coupled to a detectable label. In some instances, the detectable labels may be coupled to a 3′ nucleotide of a corresponding dinucleotide molecule.

In some aspects, the detectable moiety is a fluorophore. In some instances, the fluorophores may be conjugated to a nucleobase moiety of the 3′ nucleotide in a corresponding dinucleotide molecule.Examples of fluorescent labels and nucleotides and/or polynucleotides conjugated to such fluorescent labels comprise those described elsewhere herein and those described in, for example, Hoagland, Handbook of Fluorescent Probes and Research Chemicals, Ninth Edition (Molecular Probes, Inc., Eugene, 2002); Keller and Manak, DNA Probes, 2^{nd} Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). In some embodiments, examples of techniques and methods methodologies applicable to the provided embodiments comprise those described in, for example, U.S. Pat. No. 4,757,141, U.S. Pat. No. 5,151,507 and U.S. Pat. No. 5,091,519. In some embodiments, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes). Labelling can also be carried out with quantum dots, as described in U.S. Pat. No. 6,322,901, U.S. Pat. No. 6,576,291, U.S. Pat. No. 6,423,551, U.S. Pat. No. 6,251,303, U.S. Pat. No. 6,319,426, U.S. Pat. No. 6,426,513, U.S. Pat. No. 6,444,143, U.S. Pat. No. 5,990,479, U.S. Pat. No. 6,207,392, US 2002/0045045 and US 2003/0017264. In some instances, a fluorescent label comprises a signaling moiety that conveys information through the fluorescence absorption and/or emission properties of one or more molecules. Examples of fluorescence properties comprise fluorescence intensity, fluorescence lifetime, emission spectrum characteristics and energy transfer.

In some aspects, a detectable signal for one of the four sets is an absence of a detectable moiety. For example, if three sets of dinucleotide molecules are each associated with a detectable label moiety, and a fourth set does not have a detectable moiety attached (i.e., is “non-labeled”), then upon imaging/detection, an absence of a signal would indicate that the shared nucleotide of the fourth set (e.g., a same 5′ nucleotide of the fourth set) paired with the interrogated base of the template nucleic acid. In some instances, non-labeled dinucleotide molecules are used to implement a two-color or a three-color detection scheme. In some instances, non-labeled dinucleotide molecules are used to implement the readout of target-specific barcode designs, to help minimize optical crowding when performing in situ sequencing (see, e.g., PCT International Patent Application Publication Nos. US 2022/0083832, US 2022/0084629, US 2022/0084628, and WO 2023/220300, each of which is incorporated herein by reference in its entirety).

In some instances, for example, the plurality of dinucleotide molecules comprises a set of sixteen different dinucleotide molecules (e.g., each position of the dinucleotide sequence independently selected from A, C, G, and at least one of T and U), where dinucleotide molecules comprising the same first (5′) nucleotide moiety (e.g., comprising the same nucleobase) are coupled to the same fluorophore, and where dinucleotide molecules comprising different first (5′) nucleotide moieties (e.g., comprising different nucleobases) are each coupled to a different fluorophore.

In some instances, the plurality of dinucleotide molecules comprises a set of sixteen different dinucleotide molecules (e.g., selected from pairwise combinations of A, C, G, and either T or U), where the dinucleotide molecules of three of the four sets of dinucleotide molecules (e.g., sets comprising a same first (5′) nucleotide moiety and a different second (3′) nucleotide moiety) are coupled to different fluorophores and the dinucleotide molecules of one of the sets of dinucleotide molecules are not conjugated to a fluorophore.

In some instances, the plurality of dinucleotide molecules comprises a set of sixteen different dinucleotide molecules (e.g., selected from pairwise combinations of A, C, G, and either T or U), where the dinucleotide molecules of two of the four sets of dinucleotide molecules (e.g., sets comprising a same first (5′) nucleotide moiety and a different second (3′) nucleotide moiety) are coupled to different fluorophores, the dinucleotide molecules of one of the four sets are each coupled to both of the two different fluorophores, and the dinucleotide molecules of one of the four sets are not conjugated to a fluorophore. As a non-limiting example, a first set having sequences 5′-AA-3′, 5′-AT-3′, 5′-AC-3′, and 5′-AG-3′may each be labeled with a green fluorophore, a second set having sequences 5′-TA-3′, 5′-TT-3′, 5′-TC-3′, and 5′-TG-3′ may be labeled with a red fluorophore, a third set having sequences 5′-CA-3′, 5′-CT-3′, 5′-CC-3′, and 5′-CG-3′ may be labeled with a green fluorophore and a red fluorophore, and a fourth set having sequences 5′-GA-3′, 5′-GT-3′, 5′-GC-3′, and 5′-GG-3′may be unlabeled.

In some instances, molecules of the first plurality of dinucleotide molecules and the at least one additional plurality of dinucleotide molecules each lack a reversible terminator moiety (e.g., a 3′ reversible terminator moiety).

### Other Components of Dinucleotide-Based Sequencing

In some embodiments, disclosed herein are methods that include a step of incorporating a reversibly terminated nucleotide molecule into a priming strand or an extended priming strand. In some instances, the reversibly terminated nucleotide molecules comprise any of a variety of naturally-occurring nucleotides and/or functional analogs thereof (e.g., nucleotide analogs capable of hybridizing to a nucleic acid sequence in a sequence-specific/correctly base-paired manner).

In some embodiments, a priming strand is a primer or generated from a primer. A primer is generally a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence. A primer extension reaction generally refers to any method where two nucleic acid sequences become linked (e.g., hybridized) by an overlap of their respective terminal complementary nucleic acid sequences (e.g., 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

In some instances, the 3′ reversibly terminated nucleotide may be a 3′-O-blocked reversibly terminated nucleotide. In some instances, the 3′-O-blocked reversibly terminated nucleotide may be, e.g., a 3′-O-azidomethyl deoxynucleotide triphosphate (3′-O-azidomethyl-dNTP), a 3′-O-allyl deoxynucleotide triphosphate (3′-O-allyl-dNTP), or a 3′-O-amino deoxynucleotide triphosphate (3′-O-NH₂-dNTP). In some instances, the 3′ reversibly terminated nucleotide may be a 3′-unblocked reversibly terminated nucleotide.

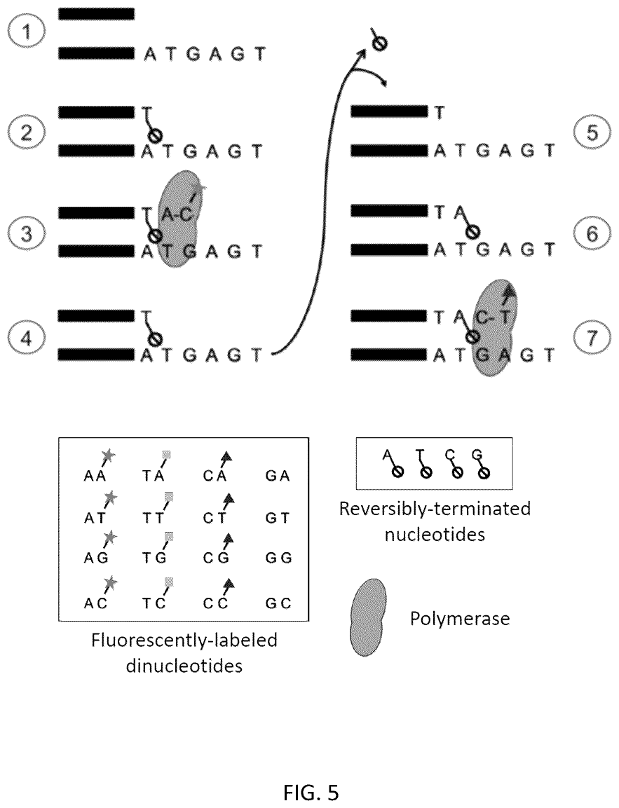

In some instances, the disclosed method for performing nucleic acid sequencing (e.g., in situ and/or flow cell sequencing) comprises the use of primer sequences that are complementary to a template nucleic acid, e.g., a subsequence (or primer binding site) that is part of an endogenous nucleic acid target sequence or a sequence (or primer binding site) that is located at or near a barcode (identifier) sequence associated with a target analyte. In some instances, a primer sequence may be designed to hybridize to a primer binding site associated with a single target analyte sequence and/or an associated target-specific barcode sequence. In some instances, a primer sequence may be designed to hybridize to a sequence (or primer binding site) that is associated with a plurality of target analyte sequences and/or associated target-specific barcode sequences (e.g., at least 2, 3, 4, 5, 6, 7, 8,9, 10, 20, 30, 40, 50, 60, 70, 80, 90, 100, 150, 200, 250, 300, 350, 400, 450, 500, 550, 600, 650, 700, 750, 800, 850, 900, 950, 1000, or more than 1000 target analyte sequences and/or associated target-specific barcode sequences). In some instances, the tissue sample is contacted directly with a primer comprising a 3′ reversibly terminated nucleotide under conditions that promote hybridization of the primer to the template nucleic acid molecule, for instance, leading directly to step 2 in FIG. 5. Alternatively, in some instances, the primer does not include a 3′ reversibly terminated nucleotide, and a reversibly terminated nucleotide molecule is incorporated into the primer strand prior to contacting the template nucleic acid with a dinucleotide molecule and a polymerase.

In some instances, the polymerase comprises, e.g., Taq polymerase, Therminator™ DNA polymerase, a Klenow fragment of DNA polymerase I, or any combination thereof. In some instances, the polymerase is not labelled with a detectable label.

In some embodiment, the methods disclosed herein include sequencing a template nucleic acid molecule that is associated with an analyte of interest. In some embodiment, the methods disclosed herein include sequencing multiple different template nucleic acid molecules, which are associated with multiple different analytes of interest. One or more analytes of interest may be derived from or present within a biological sample. In some embodiment, methods for performing multiplexed assays to analyze two or more different analytes in a single biological sample are provided.

The methods and compositions disclosed herein can be used to detect and analyze a wide variety of different analytes. In some aspects, an analyte is a nucleic acid analyte, and in some aspects, the nucleic acid analyte is sequenced directly and/or the nucleic acid analyte is associated with a reporter nucleic acid molecule that is sequenced. In some aspects, an analyte can include any biological substance, structure, moiety, or component to be analyzed. In some aspects, a target disclosed herein may similarly include any analyte of interest. In some examples, a target or analyte can be directly or indirectly detected.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of a RCA template (e.g. a padlock or other circularizable probe). Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo-or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

In some embodiments, an analyte herein is endogenous to a biological sample and can include endogenous nucleic acid analytes and non-nucleic acid analytes. Methods and compositions disclosed herein can be used to analyze nucleic acid analytes (e.g., using a nucleic acid probe or probe set that directly or indirectly hybridizes to a nucleic acid analyte) and/or non-nucleic acid analytes (e.g., using a labeling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte can be an organelle (e.g., nuclei or mitochondria). In some embodiments, the analyte is an extracellular analyte, such as a secreted analyte. Examples of analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments described herein, an analyte may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

In some embodiments, the template nucleic acid comprises a reporter nucleic acid or a portion thereof. In some embodiments, the reporter nucleic acid is for analyzing an endogenous analyte (e.g., RNA, ssDNA, cell surface or intracellular proteins, and/or metabolites). For example, the endogenous analyte may be labeled in a sample using one or more labeling agents. In some embodiments, an analyte labeling agent includes an analyte binding moiety that interacts with an analyte (e.g., an endogenous analyte in a sample). In some embodiments, the labeling agent comprises the reporter nucleic acid. In some embodiments, the labeling agent binds to the endogenous analyte and binds to the reporter nucleic acid. For example, the reporter nucleic acid comprises a barcode sequence that permits identification of the labeling agent.

In some embodiments, the method comprises one or more post-fixing (also referred to as post-fixation) steps after contacting the sample with one or more labeling agents.

In the methods and systems described herein, one or more labeling agents capable of binding to or otherwise coupling to one or more features may be used to characterize analytes, cells and/or cell features. In some instances, cell features include cell surface features. In some embodiments, a labeling agent comprises or is attached to a reporter nucleic acid that is indicative of a cell surface feature to which the analyte binding moiety of the labeling agent binds, and the reporter nucleic acid comprises a barcode sequence that permits identification of the analyte binding moiety and therefore the cell surface feature. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

An analyte binding moiety of an analyte labeling agent may include any molecule or moiety capable of binding to an analyte (e.g., a biological analyte, e.g., a macromolecular constituent). A labeling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof.

In some embodiments, the sequencing methods described herein involve identifying multiple sequences of interest, for example, associated with multiple different endogenous analytes. In some embodiments, the methods include identifying a first sequence of a first reporter oligonucleotide and identifying a second sequence of a second reporter nucleic acid. For example, a labeling agent that is specific to one type of cell feature (e.g., a first cell surface feature) may have coupled thereto a first reporter oligonucleotide, while a labeling agent that is specific to a different cell feature (e.g., a second cell surface feature) may have a different reporter oligonucleotide coupled thereto. For a description of examples of labeling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some embodiments, an analyte binding moiety includes one or more antibodies or epitope-binding fragments thereof. The antibodies or epitope-binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labeling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labeling agents are the same. In some embodiments in which the plurality of analytes includes a single species of analyte, the analyte binding moieties of the plurality of analyte labeling agents are the different (e.g., members of the plurality of analyte labeling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labeling agent that is specific to a particular cell feature may have a first plurality of the labeling agent (e.g., an antibody or lipophilic moiety) coupled to a first reporter oligonucleotide and a second plurality of the labeling agent coupled to a second reporter oligonucleotide.

In some aspects, these reporter oligonucleotides may comprise nucleic acid barcode sequences that permit identification of the labeling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using the in situ detection techniques described herein.

Attachment (coupling) of the reporter oligonucleotides to the labeling agents may be achieved through any of a variety of direct or indirect, covalent or non-covalent associations or attachments. For example, oligonucleotides may be covalently attached to a portion of a labeling agent (such a protein, e.g., an antibody or antibody fragment) using chemical conjugation techniques (e.g., Lightning-Link® antibody labeling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labeling agents. Commercially available kits, such as those from Thunderlink and Abcam, and techniques common in the art may be used to couple reporter oligonucleotides to labeling agents as appropriate. In another example, a labeling agent is indirectly (e.g., via hybridization) coupled to a reporter oligonucleotide comprising a barcode sequence that identifies the label agent. For instance, the labeling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labeling agent to the reporter oligonucleotide. In some embodiments, the reporter oligonucleotides are releasable from the labeling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein.

In some cases, the labeling agent comprises a reporter oligonucleotide and a label. A label can be fluorophore, a radioisotope, a molecule capable of a colorimetric reaction, a magnetic particle, or any other suitable molecule or compound capable of detection. The label can be conjugated to a labeling agent (or reporter oligonucleotide) either directly or indirectly (e.g., the label can be conjugated to a molecule that can bind to the labeling agent or reporter oligonucleotide). In some cases, a label is conjugated to a first oligonucleotide that is complementary (e.g., hybridizes) to a sequence of the reporter oligonucleotide.

In some embodiments, the template nucleic acid molecule includes a barcode sequence. In some embodiments, an analyte described herein can be associated with one or more barcode sequences. In some embodiments, an analyte as described herein is associated with at least two, three, four, five, six, seven, eight, nine, ten, or more barcode sequences. Barcode sequences can be used to spatially-resolve molecular components found in biological samples, for example, within a cell or a tissue sample. A barcode sequence can be attached to an analyte or to another moiety or structure (e.g., a target-specific antibody) in a reversible or irreversible manner. In some aspects, a barcode sequence comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some embodiments, a barcode sequence includes two or more sub-barcode sequences (or barcode segments) that together function as a single barcode sequence. For example, a polynucleotide barcode sequence can include two or more polynucleotide sequences (e.g., sub-barcode sequences) that are contiguous or that are separated by one or more non-barcode sequences. In some embodiments, a barcode sequence comprises about 1, 2, 3, 4, 5, 6, 7, 8, 9, 10, or more than 10 sub-barcode sequences (or barcode segments). In some embodiments, each sub-barcode sequence (or barcode segment) comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides. In some embodiments, each non-barcode sequence comprises about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some embodiments, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide. In any of the preceding embodiments, the methods provided herein can include analyzing the barcodes by performing an in situ dinucleotide-based sequencing workflow as described herein.

In some embodiments, e.g., in a barcode sequencing method, barcode sequences are detected for identification of other molecules including nucleic acid molecules (DNA or RNA) that are longer than the barcode sequences themselves, as opposed to direct sequencing of the longer nucleic acid molecules. In some embodiments, an N-mer barcode sequence comprises up to 4^{N} unique sequences given a sequencing read of N bases, and a much shorter sequencing read may be required for molecular identification compared to non-barcoded sequencing methods such as direct sequencing. For example, 1024 molecular species may be identified using a 5-nucleotide barcode sequence (4⁵=1024), whereas 8 nucleotide barcodes can be used to identify up to 65,536 molecular species, a number greater than the total number of distinct genes in the human genome. In some embodiments, the barcode sequences contained in reporter oligonucleotides, such as in adapters, probes or RCPs are detected, rather than endogenous sequences, which can be an efficient read-out in terms of information per cycle of sequencing. Because the barcode sequences are pre-determined, they can also be designed to feature error detection and correction mechanisms, see, e.g., U.S. Pat. Pub. 20190055594 and U.S. Pat. Pub 20210164039, which are hereby incorporated by reference in their entirety.

### Workflows for Performing Dinucleotide-Based In Situ or Flow Cell Sequencing

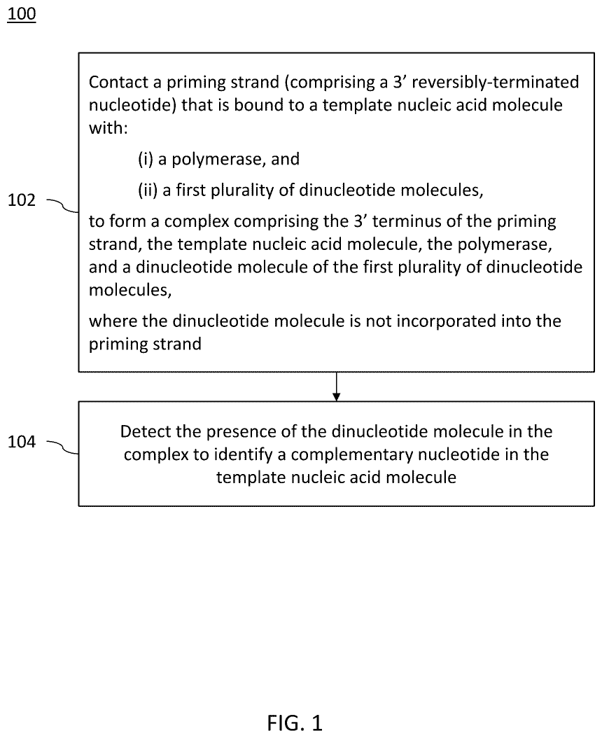

FIG. 1 provides a non-limiting example of a flowchart for a process 100 for sequencing a template nucleic acid molecule in accordance with one implementation of the methods described herein. The sequencing steps depicted in FIG. 1 may be performed as part of an in situ sequencing method or as part of a flow cell sequencing method. In process 100, some steps may optionally be combined, the order of some steps may optionally be changed, and some steps may optionally be omitted. In some instances, additional steps may be performed in combination with the steps shown in process 100. Accordingly, the steps illustrated (and described in greater detail below) for process 100 are exemplary by nature, and as such, should not be viewed as limiting.

At step 102 in FIG. 1, a priming strand (comprising a 3′ reversibly terminated nucleotide) that is bound to a template nucleic acid molecule is contacted with: (i) a polymerase, and (ii) a first plurality of dinucleotide molecules to form a complex comprising the 3′ terminus of the priming strand, the template nucleic acid molecule, the polymerase, and a dinucleotide molecule of the first plurality of dinucleotide molecules, where the dinucleotide molecule is not incorporated into the priming strand.

In some instances, the template nucleic acid molecule comprises an endogenous nucleic acid molecule (e.g., a DNA molecule, an RNA molecule, or an mRNA molecule) that has been reverse transcribed, amplified, and/or extracted from a biological sample (e.g., a cell sample or tissue sample).

In some instances, the template nucleic acid molecule comprises a barcode sequence (e.g., a nucleic acid barcode sequence) associated with a target analyte of interest (e.g., using the barcoding methods described elsewhere herein) that has been reverse transcribed, amplified, and/or extracted from a biological sample (e.g., a cell sample or tissue sample).

In some instances, the method may further comprise hybridizing a circularizable probe to a target analyte (or to a labeling agent bound to the target analyte), ligating the circularizable probe to form a circularized probe, and performing rolling circle amplification of the circularized probe to generate the template nucleic acid molecule. In some instances, for example, the circularizable probe may be a padlock probe. In some aspects, a template for rolling circle amplification is provided as a circularizable probe set (e.g., two or more nucleic acid molecules that can be ligated together to form a circular nucleic acid molecule, optionally wherein the two or more nucleic acid molecules are probes that can be ligated together upon hybridization to a target analyte or to a labeling agent bound to the target analyte). In some instances, the two or more nucleic acid molecules are ligated together using a first ligation templated by a target analyte or a labeling agent bound to the target analyte, and a second ligation templated by a splint oligonucleotide. In some instances, the method comprises hybridizing a circularizable probe set to a target analyte (or to a labeling agent bound to the target analyte), ligating the circularizable probe set to form a circularized probe, and performing rolling circle amplification of the circularized probe to generate the template nucleic acid molecule. In some aspects, the ligation is performed with gap-filling (e.g., as described elsewhere herein). In some aspects, the ligation is performed without gap-filling.

In some instances, the template nucleic acid molecule to be sequenced is a rolling circle amplification product in a biological sample or matrix. In some instances, the template nucleic acid molecule comprises multiple copies of a sequence of interest, and the methods described herein are applied to determine the sequence of interest. In some cases, the sequence of interest is 5′ and adjacent to a sequence capable of hybridizing to a priming strand (e.g., a sequencing primer). In some cases, the sequence of interest is a barcode sequence or complement thereof. In some cases, the sequence of interest is a sequence of a target nucleic acid that binds to a circularizable probe or probe set, wherein the circularizable probe or probe set is used as a template to generate the rolling circle amplification product. In some aspects, the circularizable probe or probe set comprises the complement of a target nucleic acid sequence. In some aspects, the complement of the target nucleic acid sequence is in a 3′ end portion and/or a 5′ end portion of the circularizable probe or probe set. In some aspects, the complement of the target nucleic acid sequence becomes incorporated into the circularizable probe or probe set by a gap-fill extension or ligation reaction. The rolling circle amplification using the circularized probe or probe set as a template allows for production of multiple copies of the original target nucleic acid sequence. In some aspects, at least ten, at least twenty, at least thirty, at least forty, at least fifty, at least one hundred, or at least one thousand copies of the original target nucleic acid sequence are produced by rolling circle amplification.

In some instances, the disclosed methods for performing nucleic acid sequencing (e.g., in situ and/or flow cell sequencing) comprises performing one or more steps of nucleic acid amplification or replication using one or more polymerases. Examples of polymerases that may be used for amplification include, but are not limited to, DNA polymerases (e.g., Taq DNA polymerase), RNA polymerases, and/or reverse transcriptases.

As noted elsewhere herein, examples of polymerases for use in rolling circle amplification (RCA) comprise DNA polymerases such phi29 (φ29) polymerase, Klenow fragment, Bacillus stearothermophilus DNA polymerase (Bst), T4 DNA polymerase, T7 DNA polymerase, or DNA polymerase I. In some aspects, DNA polymerases that have been engineered or mutated to have desirable characteristics can be employed. In some aspects, the polymerase is phi29 DNA polymerase.

In some instances, the template nucleic acid molecule to be sequenced is attached to a solid support, e.g., a sequencing flow cell. In some instances, the template nucleic acid molecule is sequenced in situ in a cell sample or tissue sample. In some instances, the cell sample comprises a layer of cells deposited on a substrate.

In some instances, the method further comprises providing: i) one or more reagents comprising the polymerase and the first plurality of dinucleotide molecules, and ii) the priming strand bound to the template nucleic acid molecule, where the priming strand comprises a 3′reversibly terminated nucleotide (at the 3′ terminus of the priming strand).

In some instances, the first plurality of dinucleotide molecules and/or at least one additional plurality of dinucleotide molecules comprises a same set of dinucleotide sequences. In some instances, the first plurality of dinucleotide molecules and/or at least one additional plurality of dinucleotide molecules comprises different sets of dinucleotide sequences.

In some instances, the first plurality of dinucleotide molecules and/or at least one additional plurality of dinucleotide molecules each lacks a 3′ reversible terminator moiety.

### Multi-Cycle Sequencing

In some instances, the first plurality of dinucleotide molecules and/or at least one additional plurality of dinucleotide molecules include four sets of four different dinucleotide sequences, with each set including a same 5′ nucleotide across the set and four different 3′ nucleotides, or each set including four different 5′ nucleotides and a same 3′ nucleotide across the set, wherein molecules of each set share a same detectable signal across the set.

In some instances, the multi-cycle sequencing process involves a repeating patten of cycles, with different cycles of the pattern having different sets (or combinations of sets) of the dinucleotide molecules. In some instances, the multi-cycle sequencing process involves a repeating pattern of four cycles, with a first cycle utilizing a first set of dinucleotide molecules (e.g., with a 5′ “A”), a second cycle utilizing a second set of dinucleotide molecules (e.g., with a 5′ “T”), a third cycle with a third set of dinucleotide molecules (e.g., with a 5′ “C”), and a fourth cycle with a fourth set of dinucleotide molecules (e.g., with a 5′ “G”). In some instances, the multi-cycle sequencing process involves a repeating pattern of two cycles, with a first cycle utilizing a first two of the four sets of dinucleotide molecules (e.g., a first set with a 5′ “A” and a second set with a 5′ “T”), and a second cycle using the other two of the four sets (e.g., a third set with a 5′ “C” and a second set with a 5′ “G”). In some instances, where a repeating patten of cycles is used, with different cycles of the pattern having different sets (or combinations of sets) of the dinucleotide molecules, the detectable signal for each of the four sets is a detectable moiety (e.g., a fluorophore conjugated to molecules of the set) that is unique among sets used in the same cycle.

In some instances, each cycle of the multi-cycle sequencing process involves use of four sets of dinucleotide molecules, such that each plurality of dinucleotide molecules includes all possible pairwise combinations of A, C, G, and T and/or U. In some instances, where all four sets are used in each cycle, the detectable signal for each of the four sets is a unique detectable moiety. In some instances, where all four sets are used in each cycle, the detectable signal for three of the four sets is a unique detectable moiety, and the detectable signal for a fourth of the four sets is an absence of a detectable moiety. In some embodiments, the dinucleotide molecules in the four set lack any detectable moiety. In some embodiments, one or more dinucleotide molecules in the four set are labeled with a detectable moiety, and the detectable signal for the fourth set is recorded as “dark” by not detecting any detectable moiety in the fourth set.

The (non-covalently bound) complex of the 3′ terminus of the priming strand, the template nucleic acid molecule, a polymerase, and a dinucleotide molecule may comprise a transient complex that is stabilized by the presence of a dinucleotide molecule. In some instances, the transient complex persists for at least 5 sec, 10 sec, 20 sec, 30 sec, 40 sec, 50 sec, 1 min, 2 min, 3 min, 4 min, 5 min, or 10 min after removal of polymerase-dinucleotide mixture used to contact the primed template nucleic acid molecule and form the complex. The “persistence time” of the complex, as used herein, refers to the average length of time that the complex remains stable without significant dissociation of any of the components of the bound complex.

At step 104 in FIG. 1, the presence of the dinucleotide molecule in the complex is detected to identify a complementary nucleotide in the template nucleic acid molecule.

In some instances, detecting a presence of a dinucleotide molecule in the complex comprises detecting a signal associated with a detectable label coupled to the dinucleotide molecule. In some instances, detecting a presence of a dinucleotide molecule in the complex comprises detecting an absence of a signal, where the absence of signal is associated with a dinucleotide molecule that is not coupled to a detectable label, e.g., a fluorophore. In some instances, the detection step may be performed, e.g., using a fluorescence imaging technique as described elsewhere herein.

In some instances, the method (or process) depicted in FIG. 1 further comprises: c) performing a deprotection reaction to deprotect the 3′ reversibly terminated nucleotide (e.g., using Tris(2-carboxyethyl)phosphine (“TCEP”) in aqueous solution to remove 3′-O-azidomethyl groups from 3′-O-azidomethyl reversibly terminated nucleotides); and d) performing an extension reaction to incorporate a 3′ reversibly terminated nucleotide that is complementary to the identified nucleotide in the template nucleic acid molecule into an extended priming strand. Examples of other deprotection agents that may be used include NaNO₂, NaClO₄, and Tris(hydroxypropyl)phosphine (“THPP”). In some instances, the additional reversibly terminated nucleotide does not comprise a detectable label.

In some embodiments, a method disclosed herein comprises washing (e.g., by rinsing) a cell or tissue sample to remove molecules of the polymerase and unbound dinucleotide molecules prior to performing a detection step to facilitate detection of a fluorescence signal (or absence thereof) associated with the dinucleotide in the complex. In some embodiments, a method disclosed herein comprises washing a flow cell to remove molecules of the polymerase and unbound dinucleotide molecules prior to performing a detection step to facilitate detection of a fluorescence signal (or absence thereof) associated with the dinucleotide in the complex.

In some instances, the method (or process) depicted in FIG. 1 further comprises performing a first wash step to remove unbound polymerase and unbound dinucleotide molecules prior to performing the detecting step. In some embodiments, the first wash conditions are configured to not disrupt the bound complex. In some embodiments, the first wash step may comprise, for example, use of the same buffer used for contacting the primed template nucleic acid with a polymerase and dinucleotide molecules (but without the polymerase and dinucleotide molecules). In some embodiments, the first wash buffer may not include KCl and/or may include little to no DMSO. In some embodiments, the first wash buffer is similar to those used for wash buffers as used in wash steps of a Western blot (e.g., a wash buffer added in a Western blot after binding a primary antibody but washing prior to incubation with a secondary antibody, such as PBST). PBST is a phosphate-buffered saline with a low-concentration of detergent, such as 0.05% to 0.1% Tween. In some instances, the method further comprises performing a second wash step after performing the detecting step to disrupt the complex. In some embodiments, the second wash is performed under more stringent conditions than the first wash. For example, the second wash may include a temperature higher than room temperature (e.g., 30-40° C.), a higher salt concentration (e.g., a higher KCl salt concentrations (e.g., at least 50 mM KCl)), a solvent miscible in the wash buffer solution (e.g., DMSO), a detergent (e.g., sodium dodecyl sulfate (SDS)), or a combination thereof.

In some instances, the method (or process) depicted in FIG. 1 further comprises repeating steps (a)-(d) for at least one additional cycle using at least one additional plurality of dinucleotide molecules to detect a presence of a dinucleotide molecule in a complex and thereby identify at least one additional complementary nucleotide in the template nucleic acid molecule. In some instances, the at least one additional cycle comprises at least 2, 5, 10, 20, 30, 40, or 50 additional cycles.

In some instances, the method (or process) depicted in FIG. 1 further comprises: prior to performing a first contacting step in (a), hybridizing a primer that does not comprise a 3′ reversibly terminated nucleotide at its 3′ end to a primer binding site in the template nucleic acid molecule; and performing an extension reaction to incorporate a 3′ reversibly terminated nucleotide that is complementary to a nucleotide in the template nucleic acid molecule into an extended primer strand to generate the priming strand.

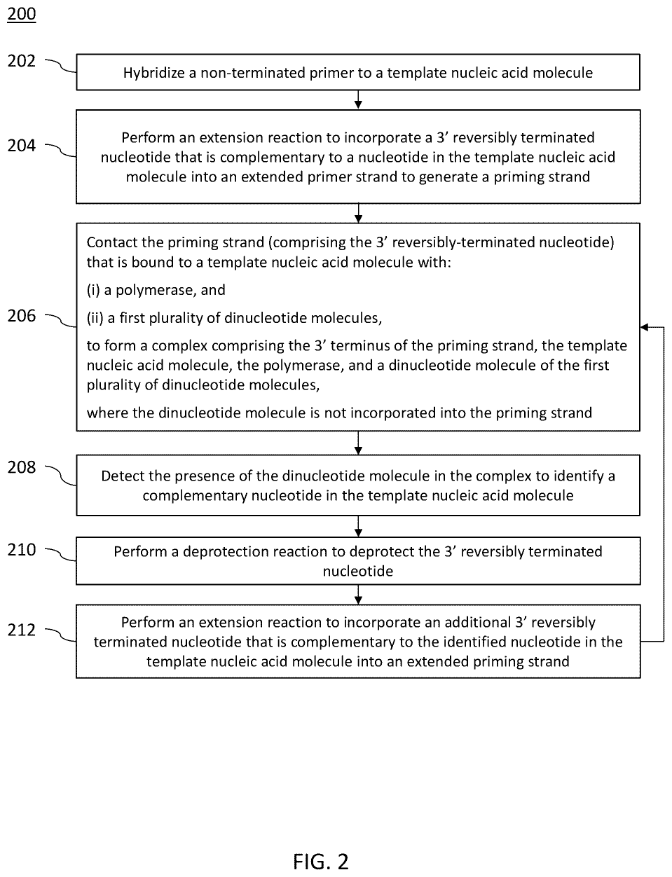

FIG. 2 provides a non-limiting example of a flowchart for a process 200 for sequencing a template nucleic acid molecule in accordance with one implementation of the methods described herein. In some aspects, the sequencing steps depicted in FIG. 2 are performed as part of an in situ sequencing method or as part of a flow cell sequencing method. In process 200, some steps may optionally be combined, the order of some steps may optionally be changed, and some steps may optionally be omitted. In some instances, additional steps are performed in combination with the steps shown in process 200. Accordingly, the steps illustrated (and described in greater detail below) for process 200 are exemplary by nature, and as such, should not be viewed as limiting.

At step 202 in FIG. 2, a non-terminated primer sequence is hybridized to a template nucleic acid molecule.

In some instances, the template nucleic acid molecule comprises an endogenous nucleic acid molecule (e.g., a DNA molecule, an RNA molecule, or an mRNA molecule) that has been reverse transcribed, amplified, and/or extracted from a biological sample (e.g., a cell sample or tissue sample).

In some instances, the template nucleic acid molecule comprises a barcode sequence (e.g., a nucleic acid barcode sequence) associated with a target analyte of interest (e.g., using the barcoding methods described elsewhere herein) that has been reverse transcribed, amplified, and/or extracted from a biological sample (e.g., a cell sample or tissue sample).

In some instances, the method may further comprise hybridizing a circularizable probe to a target analyte (or to a labeling agent bound to the target analyte), ligating the circularizable probe to form a circularized probe, and performing rolling circle amplification of the circularized probe to generate the template nucleic acid molecule. In some instances, for example, the circularizable probe may be a padlock probe.

In some instances, the template nucleic acid molecule to be sequenced may be attached to a solid support, e.g., a sequencing flow cell. In some instances, the template nucleic acid molecule is sequenced in situ in a cell sample or tissue sample. In some instances, the cell sample comprises a layer of cells deposited on a substrate.

At step 204 in FIG. 2, an extension reaction is performed to incorporate a 3′ reversibly terminated nucleotide that is complementary to a nucleotide in the template nucleic acid molecule into an extended primer strand to generate a priming strand.

In some instances, the 3′ reversibly terminated nucleotide comprises a 3′-O-blocked reversibly terminated nucleotide. In some instances, the 3′-O-blocked reversibly terminated nucleotide comprises a 3′-O-azidomethyl deoxynucleotide triphosphate (3′-O-azidomethyl-dNTP), a 3′-O-allyl deoxynucleotide triphosphate (3′-O-allyl-dNTP), or a 3′-O-amino deoxynucleotide triphosphate (3′-O-NH₂-dNTP). In some instances, the 3′ reversibly terminated nucleotide comprises a 3′-unblocked reversibly terminated nucleotide.

At step 206 in FIG. 2, the priming strand (comprising the 3′ reversibly terminated nucleotide) that is bound to a template nucleic acid molecule is contacted with: (i) a polymerase, and (ii) a first plurality of dinucleotide molecules to form a complex comprising the 3′ terminus of the priming strand, the template nucleic acid molecule, the polymerase, and a dinucleotide molecule of the first plurality of dinucleotide molecules, where the dinucleotide molecule is not incorporated into the priming strand.

In some instances, the method further comprises providing: i) one or more reagents comprising the polymerase and the first plurality of dinucleotide molecules, and ii) the priming strand bound to the template nucleic acid molecule, where the priming strand comprises a 3′ reversibly terminated nucleotide (at the 3′ terminus of the priming strand).

In some instances, the polymerase comprises, e.g., Taq polymerase, 9ºN DNA polymerase (or variant thereof, for example, D141A/E143A/A485L), a Klenow fragment of DNA polymerase I, or any combination thereof. In some instances, the polymerase is not labeled with a detectable label.

In some instances, the (non-covalently bound) complex of the 3′ terminus of the priming strand, the template nucleic acid molecule, a polymerase, and a dinucleotide molecule comprises a transient complex that is stabilized by the presence of the 3′ nucleotide of the dinucleotide molecule. In some instances, the transient complex may persist for at least 5 sec, 10 sec, 20 sec, 30 sec, 40 sec, 50 sec, 1 min, 2 min, 3 min, 4 min, 5 min, or 10 min after removal of the polymerase-dinucleotide mixture used to contact the primed template nucleic acid molecule and form the complex. The “persistence time” of the complex, as used herein, refers to the average length of time that the complex remains stable without significant dissociation of any of the components of the bound complex. In some aspects, the persistence time is measured based on the length of time a signal from a labeled dinucleotide molecule is detectable.

At step 208 in FIG. 2, the presence of the dinucleotide molecule in the complex is detected to identify a complementary nucleotide in the template nucleic acid molecule.

In some instances, detecting a presence of a dinucleotide molecule in the complex comprises detecting a signal associated with a detectable label coupled to the dinucleotide molecule. In some instances, detecting a presence of a dinucleotide molecule in the complex comprises detecting an absence of a signal, where the absence of signal is associated with a dinucleotide molecule that is not coupled to a detectable label, e.g., a fluorophore. In some instances, the detection step is performed, e.g., using a fluorescence imaging technique as described elsewhere herein.

At step 210 in FIG. 2, a deprotection reaction is performed to deprotect the 3′ reversibly terminated nucleotide (e.g., using Tris(2-carboxyethyl)phosphine in aqueous solution to remove 3′-O-azidomethyl groups from 3′-O-azidomethyl reversibly terminated nucleotides).

At step 212 in FIG. 2, an extension reaction is performed to incorporate an additional 3′ reversibly terminated nucleotide that is complementary to the identified nucleotide in the template nucleic acid molecule into an extended priming strand. In some instances, the additional reversibly terminated nucleotide does not comprise a detectable label.

In some instances, the method (or process) depicted in FIG. 2 further comprises performing a first wash step to remove unbound polymerase and unbound dinucleotide molecules prior to performing the detecting step. In some instances, the method may further comprise performing a second wash step after performing the detecting step to disrupt the complex.

As indicated in FIG. 2, steps 206 to 212 may then be repeated for at least one additional cycle using at least one additional plurality of dinucleotide molecules to detect a presence of a dinucleotide molecule in a complex and thereby identify at least one additional complementary nucleotide in the template nucleic acid molecule. In some instances, the at least one additional cycle comprises at least 2, 5, 10, 20, 30, 40, 50, or more than 50 cycles.

In some instances, the first plurality of dinucleotide molecules and at least one additional plurality of dinucleotide molecules comprise a same set of dinucleotide sequences. For example, in some instances, a first cycle uses a first plurality of dinucleotide molecules that includes four sets of four different dinucleotide sequences (amounting to sixteen different sequences), with each set including four sequences all sharing a same 5′ nucleotide (A, T/U, C, or G), a fully degenerate 3′ nucleotide (e.g., A, C, G, and T/U), and a same label; and a second cycle uses a second plurality of dinucleotide molecules that includes the same four sets of dinucleotide sequences (i.e., the same sixteen sequences).

In some instances, the first plurality of dinucleotide molecules and at least one additional plurality of dinucleotide molecules comprise different sets of dinucleotide molecules. For example, in some instances, a first cycle uses a first set of dinucleotide molecules having four different dinucleotide sequences (e.g., 5′-AT-3′, 5′-AA-3′, 5′-AC-3′, and 5′-AG-3′), a second cycle uses a second set of dinucleotide molecules having four different dinucleotide sequences (e.g., 5′-TT-3′, 5′-TA-3′, 5′-TC-3′, and 5′-TG-3′), a third cycle uses a third set of dinucleotide molecules having four different dinucleotide sequences (e.g., 5′-CT-3′, 5′-CA-3′, 5′-CC-3′, and 5′-CG-3′), and a fourth cycle uses a fourth set of dinucleotide molecules (e.g., 5′-GT-3′, 5′-GA-3′, 5′-GC-3′, and 5′-GG-3′). An another example, in some instances, a first cycle uses a first set and a second set of dinucleotide molecules (e.g., a first set having the sequences 5′-AT-3′, 5′-AA-3′, 5′-AC-3′, and 5′-AG-3′; and a second set having the sequences 5′-TT-3′, 5′-TA-3′, 5′-TC-3′, and 5′-TG-3′), and a second cycle uses a third set and a fourth set of dinucleotide molecules (e.g., a third set having the sequences 5′-CT-3′, 5′-CA-3′, 5′-CC-3′, and 5′-CG-3′, and a fourth set having the sequences 5′-GT-3′, 5′-GA-3′, 5′-GC-3′, and 5′-GG-3′).

In some instances, the first plurality of dinucleotide molecules and/or at least one additional plurality of dinucleotide molecules may each comprise sets of dinucleotide molecules that do not include a 3′ reversible terminator moiety.

In some instances, the first plurality of dinucleotide molecules and/or at least one additional plurality of dinucleotide molecules each comprises one or more sets of dinucleotide molecules, where the dinucleotide sequences in each set have a 5′ nucleotide that is the same as other dinucleotide sequences in the set and a 3′ nucleotide that is different from other dinucleotide sequences in the set. In some instances, at least one of the one or more sets of dinucleotide molecules comprise dinucleotide sequences that each have a 5′ nucleotide that is the same across the set and a 3′ nucleotide that is different from other dinucleotide sequences in the set, wherein each dinucleotide molecule of the set is coupled to a same detectable label. In some instances, the one or more sets of dinucleotide molecules comprise at least two different sets of dinucleotide molecules, where the dinucleotide sequences in a first set of the at least two different sets each have a 5′ nucleotide that is the same across the first set and that is different from a 5′ nucleotide in the other sets of the at least two different sets, and have a 3′ nucleotide that is different from other dinucleotide sequences in the first set, and where the dinucleotide molecules in different sets are coupled to different detectable labels. In some instances, one of the sets of dinucleotide molecules comprises dinucleotide molecules that are not coupled to a detectable label. In some instances, the detectable labels are coupled to a 3′ nucleotide of a corresponding dinucleotide molecule. In some instances, the detectable labels are fluorophores. In some instances, the fluorophores are conjugated to a nucleobase moiety of the 3′ nucleotide in a corresponding dinucleotide molecule.

In some instances, the first plurality of dinucleotide molecules and/or at least one additional plurality of dinucleotide molecules each comprises a set of dinucleotide molecules having four different sequences, each different sequence of the set comprising a same 5′ nucleotide across the set and a different 3′ nucleotide from other sequences in the set. In some instances, the first plurality of dinucleotide molecules and/or at least one additional plurality of dinucleotide molecules each comprises four sets of dinucleotide molecules, each of the four sets of dinucleotide molecules having four different sequences, with each different sequence of an individual set comprising a same 5′ nucleotide across the set (that differs from the other three sets) and a different 3′ nucleotide from other sequences in the set.

In some instances, the one or more sets of dinucleotide molecules, each set having four different sequences, are used in a multi-cycle sequencing process. In some instances, in each set of dinucleotide molecules used in the multi-cycle sequencing process, the dinucleotide molecules in the same set are conjugated to a same fluorophore. In some instances, the four different dinucleotide molecules of a first set used in one or more cycles of a multi-cycle sequencing process may each be conjugated to a first fluorophore that is different from a second fluorophore that is conjugated to the four dinucleotide molecules of a second set used in one or more different cycles of the multi-cycle sequencing process. In some instances, the fluorophores may be coupled to a 3′ nucleotide of a corresponding dinucleotide molecule. In some instances, the four different dinucleotide molecules in a set of dinucleotides used in one or more cycles of a multi-cycle sequencing process may not be conjugated to a fluorophore.

In some instances, the first plurality of dinucleotide molecules and/or at least one additional plurality of dinucleotide molecules each comprises four sets of dinucleotide molecules, with each of the four sets including four different dinucleotide sequences, each having a 5′ nucleotide that is the same as other dinucleotide molecules in the set and that is different from a 5′ nucleotide in dinucleotide molecules in other sets, and having a different 3′ nucleotide than other dinucleotide molecules in the set, such that the first plurality of dinucleotide molecules and at least one additional plurality of dinucleotide molecules each comprise dinucleotide molecules that include all possible pairwise combinations of A, T, C, and G. In some instances, all four sets comprises four different dinucleotide molecules that are each labeled with a same fluorophore conjugated to the 3′ nucleotide of the corresponding dinucleotide that is different from fluorophores conjugated to the four different dinucleotide molecules of the other sets. In some instances, dinucleotide molecules of three of the four sets each are labeled with a same fluorophore among the set that is different from the fluorophore of the other three sets, and dinucleotide molecules of the remaining one of the four sets are not labeled with a fluorophore.

In some instances, the 5′ nucleotide and 3′ nucleotide of a dinucleotide molecule are each independently selected from A, T, U, C, and G. In some instances, the 5′ nucleotide and 3′ nucleotide of a dinucleotide molecule are each independently selected from A, T, C, and G.

### Identifying a Nucleotide Sequence

As noted elsewhere herein, the disclosed methods for performing nucleic acid sequencing (e.g., in situ and/or flow cell sequencing) may comprise inferring the sequence of a template nucleic acid molecule from a series of optical signals (e.g., fluorescence signals) detected in images acquired during a repetitive series of sequencing reaction cycles in a process referred to as “base-calling”. The interplay of sequencing chemistry, opto-fluidics hardware, optical sensors, and signal processing software utilized in sequencing platforms affects the types of errors made during sequencing (see, e.g., Lederberger et al. (2011), “Base-calling for next-generation sequencing platforms”, Brief Bioinform. 12(5):489-497). The characterization of errors associated with the sequencing process and implementation of chemistry-, imaging-, and/or signal processing software-based methods for minimizing sequence errors are thus important for maximizing the accuracy of sequencing results.

In four-color sequencing-by-synthesis methods, for example, a set of four images—one image for each of four detection channels corresponding to the emission wavelengths for four fluorophores used to label the reversibly terminated nucleotides—are acquired in each sequencing cycle. Processing of the images to detect fluorescence intensity signals produces an intensity quadruple for the location of each sequencing colony on a flow cell surface (or the location of each target analyte, or amplified representation thereof (e.g., an RCP) in the case of in situ sequencing), where each value represents the intensity of the fluorescence signal for the detection channels corresponding to A, C, G and T. Ideally, the channel in which the maximum intensity occurs would be the base that is “called” for a given RCP or sequencing colony (or target analyte) in a given cycle. However, the chemical processes involved in sequencing are imperfect, leading to errors in base-calling (see, e.g., Cacho, et al. (2016), “A Comparison of Base-calling Algorithms for Illumina Sequencing Technology”, Briefings in Bioinformatics 17(5):786-795). In some sequencing-by-synthesis (SBS) platforms, for example, sources of error may include phasing (or lagging; e.g., where the primed template nucleic acid molecules at one or more locations fail to incorporate the next base due to variation in polymerase reaction kinetics), pre-phasing (or leading; e.g., where more than one nucleotide is incorporated in a single cycle due to, e.g., impurities in the reversibly terminated nucleotides), signal decay (due to, e.g., photobleaching and/or loss of template nucleic acid during the sequencing process), and cross-talk (e.g., when two or more fluorophore emission spectra overlap, which may cause a positive correlation between signal intensities measured in the corresponding detection channels).

A variety of statistical approaches have been developed to correct for, or minimize, such errors and generate more accurate base-calls. Examples include, but are not limited to, AYB (Goldman Group, European Molecular Biology Laboratory—European Bioinformatics Institute, Cambridgeshire, UK), and Bustard (Illumina, Inc., San Diego, CA).

The output of the base-calling process applied to optical signals detected in a series of images of a biological sample or flow cell surface acquired during a cycling sequencing process consists of a plurality of sequence reads, e.g., the nucleotide sequences determined for all or a portion of a template nucleic acid molecule (e.g., an endogenous nucleic acid analyte or a barcode sequence associated with a target analyte).

In some instances, the sequence reads generated using the disclosed methods for in situ and/or flow cell sequencing comprise sequence reads of at least about 10, 20, 30, 40, 50, 60, 70, 80, 90, 100 or more nucleotides or base pairs of the template nucleic acid sequences. In some instances, the sequence reads generated using the disclosed methods comprise sequence reads of at least about 100, 120, 140, 160, 180, 200, 220, 240, 260, 280, 300, 320, 340, 360, 380, 400, or more than 400 nucleotides or base pairs of the template nucleic acid sequences.

In some instances, the disclosed methods for in situ or flow cell sequencing may generate at least about 100, 200, 300, 400, 500, 600, 700, 800, 900, 1,000, or more sequencing reads per run. In some instances, the disclosed method may generate at least about 1,000, 1,500, 2,000, 2,500, 3,000, 3,500, 4,000, 4,500, 5,000, 5,500, 6,000, 6,500, 7,000, 7,500, 8,000, 8,500, 9,000, 9,500, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, 10⁶, 5×10⁶, 10⁷, or more than 10⁷ sequencing reads per run.

In some instances, the disclosed methods for in situ and/or flow cell sequencing comprise assembly of longer template nucleic acid sequences, e.g., genome fragments or whole genomes, from a plurality of relatively short sequence reads. Sequence assembly may be performed by identifying the overlapping sequences from multiple short sequence reads to assemble longer, contiguous sections of sequence.

In some instances, the disclosed methods for in situ and/or flow cell sequencing comprise identifying a code word corresponding to a sequence read or an assembled sequence, where the code word is one of a plurality of code words in a codebook that includes assignment of each of the plurality of code words to a target analyte of interest. The sequence read or assembled sequence may thus be used to identify a specific target analyte (based on the corresponding code word) in, e.g., a multiplexed in situ detection or sequencing assay.

In some instances, the disclosed methods for dinucleotide-based sequencing (e.g., in situ and/or flow cell sequencing) comprise alignment of sequence reads and/or assembled sequences to a known reference sequence or consensus sequence (e.g., the GRCh38 human reference genome (Genome Reference Consortium)) from the same or a similar organism. Alignment to a reference sequence or consensus sequence may be used to identify gaps, errors, or variants in the assembled sequence. Any of a variety of bioinformatics software programs known to those of skill in the art may be used to assemble longer sequences from relatively short sequence reads. Examples include, but are not limited to, DBG2OLC (see, e.g., Ye et al. (2016), “DBG2OLC: Efficient Assembly of Large Genomes Using Long Erroneous Reads of the Third Generation Sequencing Technologies”, Scientific Reports 6:31900), SPAdes (see, e.g., Bankevich et al. (2012), “SPAdes: A New Genome Assembly Algorithm and Its Applications to Single-Cell Sequencing”, J. Computational Biol. 19(5):455-477), SparseAssembler (see, e.g., Ye et al. (2012), “Exploiting Sparseness in de novo Genome Assembly”, BMC Bioinformatics 13(Suppl 6):S1), Fermi (see, e.g., Li (2012), “Exploring Single-Sample SNP and INDEL Calling with Whole-Genome de novo Assembly”, Bioinformatics 28(14):1838-1844), and String Graph Assembler (SGA) (see, e.g., Simpson et al. (2012), “Efficient de novo Assembly of Large Genomes Using Compressed Data Structures”, Genome Res. 22:549-556).

In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of the complex to identify a complementary nucleotide. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The fluorescence microscope can be or comprise any microscope that uses fluorescence to generate an image, whether it is a more simple set up like an epifluorescence microscope, or a more complicated design such as a confocal microscope, which uses optical sectioning to achieve better z-axis resolution of the sample to be imaged.

In some embodiments, confocal microscopy is used for detection and imaging of the sample. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (i.e., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immune-stained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some embodiments, a method herein comprises subjecting the sample to expansion microscopy methods and techniques. Expansion allows individual targets (e.g., mRNA or RNA transcripts) which are densely packed within a cell, to be resolved spatially in a high-throughput manner. Expansion microscopy techniques are known in the art and can be performed as described in US 2016/0116384 and Chen et al., Science, 347, 543 (2015), each of which are incorporated herein by reference in their entirety. In some embodiments, the method does not comprise subjecting the sample to expansion microscopy. In some embodiments, the method does not comprise dissociating a cell from the sample such as a tissue or the cellular microenvironment. In some embodiments, the method does not comprise lysing the sample or cells therein. In some embodiments, the method does not comprise embedding the sample or molecules from the sample in an exogenous matrix.

In some cases, analysis is performed on one or more images captured, and comprises processing the image(s) and/or quantifying signals observed. In some embodiments, images of signals from different fluorescent channels and/or nucleotide incorporation cycles can be compared and analyzed. In some embodiments, images of signals (or absence thereof) at a particular location in a sample from different fluorescent channels and/or sequential incorporation cycles can be aligned to analyze an analyte at the location. For instance, a particular location in a sample can be tracked and signal spots from sequential incorporation cycles can be analyzed to detect a target polynucleotide sequence (e.g., an endogenous nucleic acid analyte or a barcode sequence or subsequence thereof) in an analyte at the location. The analysis may comprise processing information of one or more cell types, one or more types of analytes, a number or level of analyte, and/or a number or level of cells detected in a particular region of the sample. In some embodiments, the analysis comprises detecting a sequence e.g., a barcode sequence present in an amplification product at a location in the sample. In some embodiments, the number of signals detected in a unit area in the biological sample is quantified. In some embodiments, the signals detected at a corresponding position in the biological sample in a plurality of images taken at different z positions (e.g., in the depth direction) is quantified and analyzed.

### Opto-Fluidic Instrument.

In some embodiments, the sequencing methods described herein (e.g., in situ sequencing or flow cell sequencing) include using. instruments having integrated optics and fluidics modules (“opto-fluidic instruments” or “opto-fluidic systems”) for detecting target molecules (e.g., nucleic acids, proteins, antibodies, etc.) in biological samples (e.g., one or more cells or a tissue sample) as described herein.

In an opto-fluidic instrument, the fluidics module is configured to deliver one or more reagents (e.g., detectably labeled and/or non-labeled nucleotide molecules, reversibly terminated nucleotides, primers, detectable-labeled probes and/or non-labeled probes, polymerases and/or other enzymes, deprotection reagents, buffers, etc.) to the biological sample (e.g., to a sample cartridge within which the biological sample is contained) or to a flow cell (e.g., within which nucleic acid molecules extracted from the biological sample have been tethered) and/or to remove spent reagents therefrom. In some embodiments, one or more sample preparation steps (e.g., fixing, embedding, sample clearing, and/or nucleic acid extraction (in the case that nucleic acid molecules are to be extracted and sequenced in a flow cell)) may be performed prior to the sample being placed on the instrument. In some embodiments, the fluidics module is configured to deliver one or more further reagents (e.g., primary probe(s) such as circular probe(s) or circularizable probe(s) or probe set(s)) and/or to remove non-specifically hybridized probe(s). In some embodiments, the fluidics module is configured to deliver one or more detectably labeled probes and optionally intermediate probes to detect the target analytes, or amplified representatives thereof (e.g., RCP(s)) in the biological sample. In some embodiments, the fluidics module is configured to deliver one or more nucleotide mixtures (e.g., mixtures of detectably labeled and/or non-labeled dinucleotide molecules, reversibly terminated nucleotides, as well as primers, polymerases, deprotection reagents, etc.) to sequence, e.g., native nucleic acid sequences, barcode sequences associated with target analytes, or amplified copies thereof (e.g., barcode sequences included in RCP(s)) in the biological sample. In some embodiments, the fluidics module is configured to deliver one or more nucleotide mixtures (e.g., mixtures of detectably labeled and/or non-labeled dinucleotide molecules, reversibly terminated nucleotides, as well as primers, polymerases, deprotection reagents, etc.) to a flow cell to sequence, e.g., native nucleic acid sequences, barcode sequences, or amplified copies thereof extracted from the biological sample.

Additionally, the optics module is configured to illuminate the biological sample (or flow cell) with light having one or more spectral emission curves (over a range of wavelengths) and subsequently capture one or more images of emitted light signals from the biological sample (or flow cell) during one or more decoding (e.g., probing or sequencing) cycles. In various embodiments, the captured images may be processed in real time and/or at a later time to determine the presence of the one or more target molecules in the biological sample, as well as two-dimensional and/or three-dimensional position information associated with each detected target molecule within the biological sample. In various embodiments, the captured images of a flow cell surface may be processed in real time and/or at a later time to determine the sequence of the one or more nucleic acid sequences (e.g., barcode sequences associated with one or more target molecules) that have been extracted from a biological sample. In some embodiment, the optics module further comprises an autofocus mechanism configured to maintain focus at a specified sample plane (e.g., a plane that is perpendicular to the optical axis of an objective lens of the optics module).

Additionally, the opto-fluidics instrument includes a sample module configured to receive (and, optionally, secure) one or more biological samples (e.g., biological samples contained with one or more sample cartridges), or to receive (and, optionally, secure) one or more flow cells. In some instances, the sample module includes an X-Y stage configured to move the biological sample (or flow cell) along an X-Y plane (e.g., perpendicular to the optical axis of an objective lens of the optics module).

### In Situ Dinucleotide-Based Sequencing

In some instances, the disclosed dinucleotide-based sequencing methods may be applied to in situ sequencing applications, where the dinucleotide-based sequencing reactions are substituted for conventional sequencing methods, e.g., a conventional in situ sequencing-by-synthesis (SBS) method.

The in situ sequencing methods disclosed herein may comprise performing all or a subset of the steps of:

(a) preparing the biological sample (e.g., by fixing, sectioning, embedding, and/or clearing a cell or tissue sample, as described elsewhere herein);
(b) contacting target analytes (e.g., target nucleic acid analytes and/or protein analytes) within the prepared sample with target-specific probes, as described elsewhere herein. In some instances, the target-specific probes may comprise, e.g., target-specific linear and/or circularizable nucleic acid probes (e.g., padlock probes) designed to hybridize directly or indirectly to specific target nucleic acid analytes. In some instances, the target-specific linear and/or circularizable nucleic acid probes may optionally comprise primer binding sites and/or target-specific barcode (or identifier) sequences. In some instances, the target-specific probes may comprise, e.g., target-specific antibodies designed to bind to specific target protein analytes, where the antibodies are conjugated to nucleic acid sequences. In some instances, the conjugated nucleic acid sequences may optionally comprise primer binding sites and/or target-specific barcode (or identifier) sequences;
(c) optionally performing a reverse transcription reaction (e.g., if the probed target nucleic acid analytes comprise RNA molecules) to create cDNA copies of RNA target molecules;
(d) optionally amplifying the probed target analyte molecules and/or their associated target-specific barcode sequences (e.g., using rolling circle amplification (RCA) in the case that target-specific circularizable probes were used to probe target analyte molecules and/or associated barcode sequences);
(e) contacting the optionally amplified target nucleic acid analytes and/or associated target-specific barcode sequences with sequencing primers designed to hybridize directly or indirectly to the target nucleic acid analytes and/or their associated target-specific barcode sequences. In some instances, the sequencing primers may comprise 3′ reversibly terminated nucleotides at their 3′ termini, thereby blocking the incorporation of nucleotides into the sugar-phosphate backbone of the priming strand when contacting primed template nucleic acid molecules (e.g., target nucleic acid molecules and/or associated target-specific barcode sequences) with a polymerase and a plurality of dinucleotide molecules. In some instances, the sequencing primers may comprise free 3′-hydroxyl groups at their 3′ termini, and an initial primer extension reaction may be performed to incorporate 3′ reversibly terminated nucleotides at the 3′ termini of the bound primers (i.e., the 3′ termini of the priming strands);
(f) performing a cyclic series of base-by-base sequencing reactions, where each sequencing cycle comprises:
contacting each priming strand bound to a template nucleic acid molecule (of a plurality of primed template nucleic acid molecules present within the sample) with a polymerase and a dinucleotide molecule (e.g., at least one dinucleotide molecule or a plurality of dinucleotide molecules) comprising a first (5′) nucleotide moiety and a second (3′) nucleotide moiety to form a complex comprising the 3′ terminus of the priming strand, the template nucleic acid molecule, the polymerase, and a dinucleotide molecule comprising a first (5′) nucleotide moiety and a second (3′) nucleotide moiety that are complementary to a corresponding pair of nucleotides in the template nucleic acid molecule, where the complementary dinucleotide molecule is not incorporated into the priming strand (i.e., is not incorporated into the sugar-phosphate backbone of the priming strand) because of the presence of the 3′ reversibly terminated nucleotide. In some instances the dinucleotide molecules contacted with the primed template nucleic acid molecule may comprise detectably labeled (e.g., fluorescently labeled) dinucleotide molecules; and
detecting the presence of the dinucleotide molecule in the complex to identify a complementary nucleotide in the template nucleic acid molecule. In some instances, detecting the presence of the dinucleotide molecule may comprise detecting a signal (e.g., a fluorescence signal) associated with a detectably-labeled dinucleotide molecule (e.g., a fluorescently labeled dinucleotide molecule). In some instances, detecting the presence of the dinucleotide molecule may comprise detecting an absence of signal (e.g., the dinucleotide molecule that is complementary to the nucleotide in the template nucleic acid molecule may not comprise a fluorophore or other detectable label); (g) processing optical signals (e.g., fluorescence signals) detected in images (e.g., fluorescence images) acquired during the cyclic series of base-by-base sequencing reactions to detect the presence or absence of complementary labeled dinucleotide molecules in a complex comprising the 3′ terminus of the priming strand, the template nucleic acid molecule, the polymerase, and a complementary dinucleotide molecules in each sequencing cycle at the locations of each of a plurality of template nucleic acid molecules (i.e., the locations corresponding to each of a plurality of target analyte molecules and/or their associated target-specific barcode sequences), thereby enabling inference of the nucleotide sequence of each of the plurality of template nucleic acid molecules (e.g., the plurality of target analyte molecules and/or associated target-specific barcode sequences).

In some instances, each cycle of base-by-base sequencing may further comprise a first wash step following the contacting step and prior to the detecting step to remove any unbound polymerase and dinucleotide molecules.

In some instances, each cycle of base-by-base sequencing may further comprise a second wash step following the detection step in order to displace or disrupt the complex, and remove the displaced polymerase and dinucleotide molecule.

In some instances, each cycle of base-by-base sequencing may further comprise deprotecting the 3′ reversibly terminated nucleotides at the 3′ termini of the priming strands, and performing a primer extension reaction to incorporate cognate 3′ reversibly terminated nucleotides, thereby generating extended priming strands for each of the plurality of template nucleic acid molecules.

In some instances the detection step may comprise the use of an optical imaging technique (e.g., a fluorescence imaging technique) and real time or post-processing measurement of optical signals (e.g., fluorescence signals or the absence thereof) associated with the presence of a specific dinucleotide molecule in the complex in each sequencing cycle at a plurality of locations corresponding to a plurality of target analytes distributed throughout the biological sample.

A sample disclosed herein can be or derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium or an archaea, or may be obtained from a virus or a viroid. A biological sample can be obtained from non-mammalian organisms (e.g., a plant, an insect, an arachnid, a nematode, a fungus, or an amphibian). A biological sample can be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei), and in particular, nucleic acids (such as DNA or RNA). The biological sample can also include proteins/polypeptides, carbohydrates, and/or lipids. The biological sample can be obtained as a tissue sample, such as a tissue section, biopsy, a core biopsy, a cell pellet, a cell block, a needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a substrate.

Biological samples can be derived from a homogeneous culture or population of the subjects or organisms mentioned herein or alternatively from a collection of several different organisms. Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

In some embodiments, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety (ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). For example, in in situ sequencing embodiments, the one or more physical properties can be characterized by imaging the cell. The cell can be bound by an analyte labeling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

A substrate herein can be any support that is insoluble in aqueous liquid and which allows for positioning of biological samples, analytes, features, and/or reagents (e.g., probes) on the support. In some embodiments, a substrate is a planar substrate. In some embodiments, a substrate comprises microstructures, including protrusions and/or indentations, and the microstructures are arranged in a pattern. In some embodiments, a substrate comprises microwells. In some embodiments, a substrate is a glass substrate or a plastic substrate, such as a slide or coverslip. In some embodiments, a biological sample is attached to a substrate. Attachment of the biological sample can be irreversible or reversible, depending upon the nature of the sample and subsequent steps in the analytical method. In certain embodiments, the sample is attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose. In some embodiments, the substrate is be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick. More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

In some embodiments, the biological sample (e.g., a tissue section as described above) is prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

In some embodiments, the biological sample is a formalin-fixation and paraffin-embedding (FFPE) sample. In some embodiments, the biological sample is an FFPE tissue sample. In some embodiments, the biological sample is an FFPE tissue section. In some embodiments, the cell or tissue sample is prepared using formalin-fixation and paraffin-embedding. In some embodiments, cell suspensions and other non-tissue samples are prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, preparing a sample for in situ sequencing includes de-crosslinking a reversibly cross-linked biological sample. The de-crosslinking does not need to be complete. In some embodiments, only a portion of crosslinked molecules in the reversibly cross-linked biological sample are de-crosslinked and allowed to migrate.

In some embodiments, a biological sample can be permeabilized to facilitate transfer of species (such as probes) into the sample. If a sample is not permeabilized sufficiently, the transfer of species (such as probes) into the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample can be incubated with a cellular permeabilizing agent to facilitate permeabilization of the sample. Any suitable method for sample permeabilization can generally be used in connection with the samples described herein.

In some embodiments, the biological sample can be permeabilized by any suitable methods. For example, one or more lysis reagents can be added to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, an in situ sequencing method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

In some embodiments, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample. Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix.

A biological sample can be embedded in any of a variety of other embedding materials to provide structural substrate to the sample prior to sectioning and other handling steps. In some cases, the embedding material is removed e.g., prior to analysis of tissue sections obtained from the sample. Suitable embedding materials include, but are not limited to, waxes, resins (e.g., methacrylate resins), epoxies, and agar.

In some embodiments, the biological sample is immobilized in the hydrogel via cross-linking of the polymer material that forms the hydrogel. Cross-linking can be performed chemically and/or photochemically, or alternatively by any other suitable hydrogel-formation method.

In some embodiments, the biological sample is reversibly cross-linked prior to or during an in situ assay. In some aspects, the analytes, polynucleotides and/or amplification product (e.g., amplicon) of an analyte or a probe bound thereto can be anchored to a polymer matrix. For example, the polymer matrix can be a hydrogel. In some embodiments, one or more of the polynucleotide probe(s) and/or amplification product (e.g., amplicon) thereof can be modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible or irreversible crosslinking of the mRNA molecules.

A hydrogel may include a macromolecular polymer gel including a network. Within the network, some polymer chains can optionally be cross-linked, although cross-linking does not always occur.

In some embodiments, a hydrogel includes hydrogel subunits, such as, acrylamide, bis-acrylamide, polyacrylamide and derivatives thereof, poly (ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly (hydroxyethyl acrylate), and poly (hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

In some embodiments, the hydrogel forms the substrate. In some embodiments, the substrate comprises a hydrogel. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

To facilitate visualization, biological samples can be stained using a wide variety of stains and staining techniques. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample can be segmented using one or more images taken of the stained sample.

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples can be destained. Any suitable methods of destaining or discoloring a biological sample may be utilized and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer.

In some embodiments, in situ sequencing methods described herein include use of an opto-fluidic instrument. In various embodiments, the opto-fluidic instrument is configured to analyze one or more target molecules (e.g., one or more target RNAs) in their naturally occurring place (i.e., in situ) within the biological sample. In some embodiments, the opto-fluidic instrument is configured to analyze one or more target RNAs in relative spatial locations within the biological sample. For example, an opto-fluidic instrument may be an in-situ analysis system used to analyze a biological sample and detect target molecules including, but not limited to, DNA, RNA, proteins, antibodies, and/or the like. In some embodiments, the in situ analysis system is used to detect one or more target RNAs using target-primed rolling circle amplification (RCA) according to the methods disclosed herein.

In various embodiments, the opto-fluidic instrument may be configured to perform in situ target molecule detection via base-by-base sequencing (e.g., by sequencing an identifier sequence such as a barcode sequence associated with a target molecule) and/or any imaging or target molecule detection technique. That is, for example, an opto-fluidic instrument may include a fluidics module that includes fluids needed for establishing the experimental conditions required for the probing or sequencing of target molecules (or associate barcode sequences) in the sample. Further, such an opto-fluidic instrument may also include a sample module configured to receive the sample, and an optics module including an imaging system for illuminating (e.g., exciting one or more fluorescent probes within the sample) and/or imaging light signals received from the probed sample.

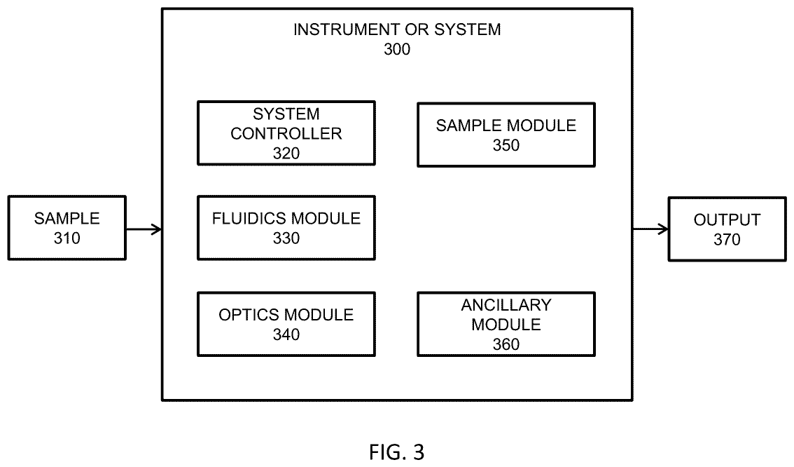

FIG. 3 shows an example of an in situ-based dinucleotide sequencing workflow to analyze target molecules of a biological sample 310 (e.g., cell or tissue sample) using an opto-fluidic instrument or system 300, according to various embodiments. In various embodiments, the sample 310 can be a biological sample (e.g., a tissue) that includes molecules such as DNA, RNA, proteins, antibodies, etc. The template nucleic acid molecule sequenced in the workflow may be a DNA or RNA molecule endogenous to the biological sample or alternatively, the template nucleic acid molecule may be a reporter nucleic acid (e.g., a barcode sequence or reporter oligo) associated with an endogenous analyte of the biological sample. For example, the sample 310 can be a sectioned tissue that is treated to access the RNA thereof for probe (e.g., circularizable probe) hybridization and sequencing (e.g., using a sequencing primer that hybridizes to RCPs to sequence barcode sequences in the RCPs) described elsewhere herein.

In various embodiments, the sample 310 may be placed in the opto-fluidic instrument or system 300 for analysis and detection of the molecules in the sample 310. In various embodiments, the opto-fluidic instrument or system 300 can be a system configured to facilitate the experimental conditions conducive for the detection of the target molecules. For example, the opto-fluidic instrument or system 300 can include a fluidics module 330, an optics module 340, a sample module 350, and an ancillary module 360, and these modules may be operated by a system controller 320 to create the experimental conditions base-by-base sequencing of nucleic acid molecules in the sample 310, as well as to facilitate the imaging of the sample (e.g., by an imaging system of the optics module 340). In various embodiments, the various modules of the opto-fluidic instrument or system 300 may be separate components in communication with each other, or at least some of them may be integrated together.

In various embodiments, the sample module 350 may be configured to receive the sample 310 into the opto-fluidic instrument or system 300. For instance, the sample module 360 may include a sample interface module (SIM) that is configured to receive a sample device (e.g., cassette) onto which the sample 310 can be deposited. That is, the sample 310 may be placed in the opto-fluidic instrument or system 300 by depositing the sample 310 (e.g., the sectioned tissue) on a sample device that is then inserted into the SIM of the sample module 350. In some instances, the sample module 350 may also include an X-Y stage onto which the SIM is mounted. The X-Y stage may be configured to move the SIM mounted thereon (e.g., and as such the sample device containing the sample 310 inserted therein) in perpendicular directions along the two-dimensional (2D) plane of the opto-fluidic instrument or system 300.

The experimental conditions that are conducive for the detection of the molecules in the sample 310 may depend on the target molecule detection technique that is employed by the opto-fluidic instrument or system 300. For example, in various embodiments, the opto-fluidic instrument or system 300 can be a system that is configured to detect target molecules (e.g., by sequencing a DNA or RNA target molecule directly and/or indirectly by sequencing an identifier sequence associated with a target molecule as a template) in the sample 310.

In various embodiments, the fluidics module 330 may include one or more components that may be used for storing the reagents, as well as for transporting said reagents to and from the sample device containing the sample 310. For example, the fluidics module 330 may include reservoirs configured to store the reagents, as well as a waste container configured for collecting the reagents (e.g., and other waste) after use by the opto-fluidic instrument or system 300 to analyze and detect the molecules of the sample 310. Further, the fluidics module 330 may also include pumps, tubes, pipettes, etc., that are configured to facilitate the transport of the reagent to the sample device (e.g., and as such the sample 310). For instance, the fluidics module 330 may include pumps (“reagent pumps”) that are configured to pump washing/stripping reagents to the sample device for use in washing/stripping the sample 310 (e.g., as well as other washing functions such as washing an objective lens of the imaging system of the optics module 340).

In various embodiments, the ancillary module 360 can be a cooling system of the opto-fluidic instrument or system 300, and the cooling system may include a network of coolant-carrying tubes that are configured to transport coolants to various modules of the opto-fluidic instrument or system 300 for regulating the temperatures thereof. In such cases, the fluidics module 330 may include coolant reservoirs for storing the coolants and pumps (e.g., “coolant pumps”) for generating a pressure differential, thereby forcing the coolants to flow from the reservoirs to the various modules of the opto-fluidic instrument or system 300 via the coolant-carrying tubes. In some instances, the fluidics module 330 may include returning coolant reservoirs that may be configured to receive and store returning coolants, e.g., heated coolants flowing back into the returning coolant reservoirs after absorbing heat discharged by the various modules of the opto-fluidic instrument or system 300. In such cases, the fluidics module 330 may also include cooling fans that are configured to force air (e.g., cool and/or ambient air) into the returning coolant reservoirs to cool the heated coolants stored therein. In some instance, the fluidics module 330 may also include cooling fans that are configured to force air directly into a component of the opto-fluidic instrument or system 300 so as to cool said component. For example, the fluidics module 330 may include cooling fans that are configured to direct cool or ambient air into the system controller 320 to cool the same.

As discussed above, the opto-fluidic instrument or system 300 may include an optics module 340 which include the various optical components of the opto-fluidic instrument or system 300, such as but not limited to a camera, an illumination module (e.g., LEDs), an objective lens, and/or the like. The optics module 340 may include a fluorescence imaging system that is configured to image the fluorescence emitted by the detectably labeled dinucleotides after the detectable labels are excited by light from the illumination module of the optics module 340.

In some instances, the optics module 340 may also include an optical frame onto which the camera, the illumination module, and/or the X-Y stage of the sample module 350 may be mounted.

In various embodiments, the system controller 320 may be configured to control the operations of the opto-fluidic instrument or system 300 (e.g., and the operations of one or more modules thereof). In some instances, the system controller 320 may take various forms, including a processor, a single computer (or computer system), or multiple computers in communication with each other. In various embodiments, the system controller 320 may be communicatively coupled with data storage, set of input devices, display system, or a combination thereof. In some cases, some or all of these components may be considered to be part of or otherwise integrated with the system controller 320, may be separate components in communication with each other, or may be integrated together. In other examples, the system controller 320 can be, or may be in communication with, a cloud computing platform.

In various embodiments, the opto-fluidic instrument or system 300 may analyze the sample 310 and may generate the output 370 that includes indications of the presence of the target molecules in the sample 310. For instance, with respect to embodiments discussed above where the opto-fluidic instrument or system 300 employs a sequencing technique for detecting molecules, the opto-fluidic instrument or system 300 may cause the sample 310 to undergo successive sequencing cycles, where during the same sequencing cycle the sample is imaged to detect signals associated with nucleotide binding and/or incorporation events at some locations in the sample 310, as well as to detect an absence of signals at other locations in the sample. In such cases, the output 370 may include a series of optical signals (e.g., a code word) specific to each identifier sequence (e.g., a barcode sequence), which allow the identification of the target molecules.

In some embodiments, provided herein are methods and compositions for analyzing one or more products of an endogenous analyte and/or a labeling agent in a biological sample. In some embodiments, an endogenous analyte (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product such as a rolling circle amplification (RCA) product) thereof is a template nucleic acid molecule analyzed by a dinucleotide-based sequencing workflow as described herein. In some embodiments, a labeling agent that directly or indirectly binds to an analyte in the biological sample is the template nucleic acid molecule. In some embodiments, a product (e.g., a hybridization product, a ligation product, an extension product (e.g., by a DNA or RNA polymerase), a replication product, a transcription/reverse transcription product, and/or an amplification product such as a rolling circle amplification (RCA) product) of a labeling agent that directly or indirectly binds to an analyte in the biological sample is analyzed.

In some embodiments, a hybridization product comprising the pairing of substantially complementary or complementary nucleic acid sequences within two different molecules can be analyzed. For example, hybridization of an endogenous analyte or the labeling agent (e.g., reporter oligonucleotide attached thereto) with another endogenous molecule or another labeling agent or a probe can be analyzed. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

Various probes and probe sets can be hybridized to an endogenous analyte and/or a labeling agent and each probe may comprise one or more barcode sequences. Examples of barcoded probes or probe sets may be based on a padlock probe, a gapped padlock probe, a SNAIL (Splint Nucleotide Assisted Intramolecular Ligation) probe set, a PLAYR (Proximity Ligation Assay for RNA) probe set, a PLISH (Proximity Ligation in situ Hybridization) probe set, and RNA-templated ligation probes. The specific probe or probe set design can vary.

In some embodiments, a ligation product of an endogenous analyte and/or a labeling agent can be analyzed. In some embodiments, the ligation product is formed between two or more endogenous analytes. In some embodiments, the ligation product is formed between two or more labeling agents. In some embodiments, the ligation product is an intramolecular ligation of an endogenous analyte. In some embodiments, the ligation product is an intramolecular ligation product or an intermolecular ligation product, for example, the ligation product can be generated by the circularization of a circularizable probe or probe set upon hybridization to a target sequence. The target sequence can be comprised in an endogenous analyte (e.g., nucleic acid such as a genomic DNA or mRNA) or a product thereof (e.g., cDNA from a cellular mRNA transcript), or in a labeling agent (e.g., the reporter oligonucleotide) or a product thereof.

In some embodiments, provided herein is a probe or probe set capable of DNA-templated ligation, such as from a cDNA molecule. See, e.g., U.S. Pat. No. 8,551,710, which is hereby incorporated by reference in its entirety. In some embodiments, provided herein is a probe or probe set capable of RNA-templated ligation. See, e.g., U.S. Pat. Pub. 2020/0224244 which is hereby incorporated by reference in its entirety. In some embodiments, the probe set is a SNAIL probe set. See, e.g., U.S. Pat. Pub. 20190055594, which is hereby incorporated by reference in its entirety. In some embodiments, provided herein is a multiplexed proximity ligation assay. See, e.g., U.S. Pat. Pub. 20140194311 which is hereby incorporated by reference in its entirety. In some embodiments, provided herein is a probe or probe set capable of proximity ligation, for instance a proximity ligation assay for RNA (e.g., PLAYR) probe set. See, e.g., U.S. Pat. Pub. 20160108458, which is hereby incorporated by reference in its entirety. In some embodiments, a circular probe can be indirectly hybridized to the target nucleic acid. In some embodiments, the circular construct is formed from a probe set capable of proximity ligation, for instance a proximity ligation in situ hybridization (PLISH) probe set. See, e.g., U.S. Pat. Pub. 2020/0224243 which is hereby incorporated by reference in its entirety.

In some embodiments, the ligation involves chemical ligation (e.g., click chemistry ligation). In some embodiments, the chemical ligation involves template dependent ligation. In some embodiments, the chemical ligation involves template independent ligation. In some embodiments, the click reaction is a template-independent reaction (see, e.g., Xiong and Seela (2011), J. Org. Chem. 76 (14): 5584-5597, incorporated by reference herein in its entirety). In some embodiments, the click reaction is a template-dependent reaction or template-directed reaction. In some embodiments, the template-dependent reaction is sensitive to base pair mismatches such that reaction rate is significantly higher for matched versus unmatched templates. In some embodiments, the click reaction is a nucleophilic addition template-dependent reaction. In some embodiments, the click reaction is a cyclopropane-tetrazine template-dependent reaction.

In some embodiments, the ligation involves enzymatic ligation. In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9º N) DNA ligase (9ºN™ DNA ligase, New England Biolabs), Taq DNA ligase, AmpligaseTM (Epicentre Biotechnologies) and phage ligases such as T3 DNA ligase, T4 DNA ligase and T7 DNA ligase and mutants thereof. In some embodiments, the ligase is a T4 RNA ligase.

In some embodiments, the ligation herein is a direct ligation. In some embodiments, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, i.e., separated by one or more intervening nucleotides or “gaps”. In some embodiments, said ends are not ligated directly to each other, but instead occurs either via the intermediacy of one or more intervening (so-called “gap” or “gap-filling” (oligo) nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo) nucleotide(s) which are complementary to a splint, padlock probe, or target nucleic acid. The gap may be a gap of 1 to 60 nucleotides or a gap of 1 to 40 nucleotides or a gap of 3 to 40 nucleotides.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tm) of the DNA strands. This selectively reduces the concentration of annealed mismatched substrates (expected to have a slightly lower Tm around the mismatch) over annealed fully base-paired substrates.

In some embodiments, the ligation herein is a proximity ligation of ligating two (or more) nucleic acid sequences that are in proximity with each other, e.g., through enzymatic means (e.g., a ligase). In some embodiments, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference). A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

In some embodiments, a primer extension product of an analyte, a labeling agent, a probe or probe set bound to the analyte (e.g., a circularizable probe bound to genomic DNA, mRNA, or cDNA), or a probe or probe set bound to the labeling agent (e.g., a circularizable probe bound to one or more reporter oligonucleotides from the same or different labeling agents) can be analyzed as the template nucleic acid molecule of the dinucleotide-based sequencing workflow.

In some embodiments, a template nucleic acid molecule of the dinucleotide-based sequencing is a product of an endogenous analyte and/or a labeling agent associated with the endogenous analyte. In some embodiments, a product of an endogenous analyte and/or a labeling agent is an amplification product of one or more polynucleotides, for instance, a circular probe or circularizable probe or probe set. In some embodiments, the amplifying is achieved by performing rolling circle amplification (RCA). In other embodiments, a primer that hybridizes to the circular probe or circularized probe is added and used as such for amplification. In some embodiments, the RCA comprises a linear RCA, a branched RCA, a dendritic RCA, or any combination thereof.

In some embodiments, the amplification is performed at a temperature between or between about 20° C. and about 60° C. In some embodiments, the amplification is performed at a temperature between or between about 30° C. and about 40° C.

In some embodiments, upon addition of a DNA polymerase in the presence of appropriate dNTP precursors and other cofactors, a primer is elongated to produce multiple copies of the circular template. This amplification step can utilize isothermal amplification or non-isothermal amplification. In some embodiments, after the formation of the hybridization complex and association of the amplification probe, the hybridization complex is rolling-circle amplified to generate a cDNA nanoball (i.e., amplicon) containing multiple copies of the cDNA. Techniques for rolling circle amplification (RCA) include linear RCA, a branched RCA, a dendritic RCA, or any combination thereof. (See, e.g., Baner et al, Nucleic Acids Research, 26:5073-5078, 1998; Lizardi et al, Nature Genetics 19:226, 1998; Mohsen et al., Acc Chem Res. 2016 Nov. 15; 49(11): 2540-2550; Schweitzer et al. Proc. Natl Acad. Sci. USA 97:101 13-1 19, 2000; Faruqi et al, BMC Genomics 2:4, 2000; Nallur et al, Nucl. Acids Res. 29: el 18, 2001; Dean et al. Genome Res. 11:1095-1099, 2001; Schweitzer et al, Nature Biotech. 20:359-365, 2002; U.S. Pat. Nos. 6,054,274, 6,291,187, 6,323,009, 6,344,329 and 6,368,801). Examples of polymerases for use in RCA comprise DNA polymerase such phi29 (φ29) polymerase, Klenow fragment, Bacillus stearothermophilus DNA polymerase (BST), T4 DNA polymerase, T7 DNA polymerase, or DNA polymerase I. In some embodiments, the polymerase is phi29 DNA polymerase.

In some aspects, during the amplification step, modified nucleotides are added to the reaction to incorporate the modified nucleotides in the amplification product (e.g., nanoball). Examples of the modified nucleotides comprise amine-modified nucleotides. In some aspects, the methods comprise anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide.

In some aspects, the polynucleotides and/or amplification product (e.g., amplicon) can be anchored to a polymer matrix. For example, the polymer matrix can be a hydrogel. In some embodiments, one or more of the polynucleotide probe(s) can be modified to contain functional groups that can be used as an anchoring site to attach the polynucleotide probes and/or amplification product to a polymer matrix. Examples of modification and polymer matrix that can be employed in accordance with the provided embodiments comprise those described in, for example, U.S. Pat. Nos. 10,138,509, 10,266,888, US 2016/0024555, US 2018/0251833 and US 2017/0219465, which are herein incorporated by reference in their entireties. In some examples, the scaffold also contains modifications or functional groups that can react with or incorporate the modifications or functional groups of the probe set or amplification product. In some examples, the scaffold comprises oligonucleotides, polymers or chemical groups, to provide a matrix and/or support structures.

The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some embodiments, the provided in situ dinucleotide-based sequencing methods involve embedding the one or more polynucleotide probe sets and/or the amplification products in the presence of hydrogel subunits to form one or more hydrogel-embedded amplification products. In some embodiments, the hydrogel-tissue chemistry comprises covalently attaching nucleic acids to in situ synthesized hydrogel for tissue clearing, enzyme diffusion, and multiple-cycle sequencing. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

In some embodiments, the RCA template may comprise the target analyte, or a part thereof, where the target analyte is a nucleic acid, or it may be provided or generated as a proxy, or a marker, for the analyte. In some embodiments, different analytes are detected in situ in one or more cells using a RCA-based detection system, e.g., where the signal is provided by generating an RCA product from a circular RCA template which is provided or generated in the assay, and the RCA product is sequenced by a dinucleotide-based workflow as described herein, to detect the corresponding analyte. The RCA product may thus be regarded as a reporter which is detected to detect the target analyte. However, the RCA template may also be regarded as a reporter for the target analyte; the RCA product is generated based on the RCA template, and comprises complementary copies of the RCA template. The RCA template determines the signal which is detected, and is thus indicative of the target analyte. As will be described in more detail below, the RCA template may be a probe, or a part or component of a probe, or may be generated from a probe. The RCA template used to generate the RCP may thus be a circular (e.g. circularized) reporter nucleic acid molecule, namely from any RCA-based detection assay which uses or generates a circular nucleic acid molecule as a reporter for the assay. Since the RCA template generates the RCP and the RCP is sequenced by a dinucleotide-based workflow as described herein, sequencing the RCP can be used to detect the target analyte corresponding to the RCA template and the RCP.

In some embodiments, a product herein includes a molecule or a complex generated in a series of reactions, e.g., hybridization, ligation, extension, replication, transcription/reverse transcription, and/or amplification (e.g., rolling circle amplification), in any suitable combination.

In some embodiments, provided herein is method of detecting a complementary nucleotide in situ using dinucleotide sequencing disclosed herein. In some embodiments, the detecting comprises detecting a presence or absence of a fluorescent signal. Fluorescence detection in tissue samples can often be hindered by the presence of strong background fluorescence. “Autofluorescence” is the general term used to distinguish background fluorescence (that can arise from a variety of sources, including aldehyde fixation, extracellular matrix components, red blood cells, lipofuscin, and the like) from the desired immunofluorescence from the fluorescently labeled antibodies or probes. Tissue autofluorescence can lead to difficulties in distinguishing the signals. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (Max Vision Biosciences), and/or a very intense black dye (e.g., Sudan Black, or comparable dark chromophore).

### Dinucleotide-Based Flow Cell Sequencing

In some instances, the disclosed dinucleotide-based sequencing methods may be applied to flow cell sequencing applications, where the dinucleotide-based sequencing reactions are substituted for the stepwise nucleotide incorporation reactions used to probe a template nucleic acid sequence in, e.g., a conventional flow cell sequencing method.

The flow cell sequencing methods disclosed herein may comprise performing all or a subset of the steps of:

(a) extraction and purification of nucleic acid molecules (e.g., endogenous nucleic acid molecules) from a biological sample, as described elsewhere herein;
(b) preparation of a sequencing library comprising template nucleic acid molecules (e.g., the endogenous nucleic acid molecules or fragments thereof) that have been end-repaired and ligated to adapter sequences, as described elsewhere herein;
(c) optionally performing nucleic acid amplification of all or a portion of the sequencing library, as described elsewhere herein;
(d) immobilizing the template nucleic acid molecules (e.g., denatured, single-stranded template nucleic acid molecules) from the sequencing library on an inner surface of a flow cell using capture probes (e.g., complementary adapter sequences) that have been tethered to the flow cell surface;
(e) performing clonal amplification of the immobilized template nucleic acid molecules to create clusters comprising, e.g., hundreds or thousands of copies of the template nucleic acid molecule immobilized at each of a plurality of locations on the flow cells surface;
(f) contacting the template nucleic acid molecules in each clonally amplified cluster with sequencing primers designed to hybridize to, e.g., the adapter sequences ligated to the template nucleic acid molecules. In some instances, the sequencing primers may comprise 3′ reversibly terminated nucleotides, thereby blocking the incorporation of dinucleotide molecules into the sugar-phosphate backbone of the priming strand when contacting primed template nucleic acid molecules with a polymerase and a plurality of dinucleotide molecules. In some instances, the sequencing primers may comprise free 3′-hydroxyl groups at their 3′ termini, and an initial primer extension reaction may be performed to incorporate 3′ reversibly terminated nucleotides at the 3′ termini of the bound primers (i.e., the 3′ termini of the priming strands);
(g) performing a cyclic series of base-by-base sequencing reactions, where each sequencing cycle comprises:
contacting each priming strand bound to a template nucleic acid molecule (of a plurality of primed template nucleic acid molecules immobilized on the surface of the flow cell) with a polymerase and a dinucleotide molecule (e.g., at least one dinucleotide molecule or a plurality of dinucleotide molecules) to form a complex comprising the 3′ terminus of the priming strand, the template nucleic acid molecule, the polymerase, and a dinucleotide molecule that is complementary to a pair of nucleotides in the template nucleic acid molecule, where the dinucleotide molecule is not incorporated into the priming strand (i.e., is not incorporated into the sugar-phosphate backbone of the priming strand) because of the presence of the 3′ reversibly terminated nucleotide. In some instances, the at least one dinucleotide molecule or the plurality of dinucleotide molecules may further comprise detectably labeled (e.g., fluorescently labeled) dinucleotide molecules; and
detecting the presence of the dinucleotide molecule in the complex to identify a complementary nucleotide in the template nucleic acid molecule. In some instances, detecting the presence of the dinucleotide molecule may comprise detecting a signal (e.g., a fluorescence signal) associated with a detectably-labeled dinucleotide molecule (e.g., a fluorescently labeled dinucleotide molecule). In some instances, detecting the presence of the dinucleotide molecule may comprise detecting an absence of signal (e.g., the dinucleotide molecule that is complementary to the nucleotide in the template nucleic acid molecule may not comprise a fluorophore or other detectable label);
   (h) processing optical signals (e.g., fluorescence signals) detected in images (e.g., fluorescence images) acquired during the cyclic series of base-by-base sequencing reactions to detect the presence or absence of complementary fluorescently labeled dinucleotide molecules in a complex comprising the 3′ terminus of the priming strand, the template nucleic acid molecule, the polymerase, and a complementary dinucleotide molecules in each sequencing cycle at the locations of each of a plurality of template nucleic acid molecules (i.e., the locations corresponding to each of a plurality of target analyte molecules and/or their associated target-specific barcode sequences), thereby enabling inference of the nucleotide sequence of each of the plurality of template nucleic acid molecules.

In some instances, each cycle of base-by-base sequencing may further comprise a first wash step prior to the detecting step to remove unbound polymerase and dinucleotide molecules.

In some instances, each cycle of base-by-base sequencing may further comprise a second wash step following the detection step in order to disrupt the complex and remove the displaced polymerase and dinucleotide molecule.

In some instances, each cycle of base-by-base sequencing may further comprise deprotecting the 3′ reversibly terminated nucleotides at the 3′ termini of the priming strands, and performing a primer extension reaction to incorporate cognate 3′ reversibly terminated nucleotides, thereby generating extended priming strands for each of the plurality of template nucleic acid molecules.

In some instances the detection step may comprise the use of an optical imaging technique (e.g., a fluorescence imaging technique) and real time or post-processing measurement of optical signals (e.g., fluorescence signals or the absence thereof) associated with the presence of a specific dinucleotide molecule in the complex in each sequencing cycle at a plurality of locations corresponding to a plurality of template nucleic acid molecules distributed across the flow cell surface.

In some aspects, provided herein are dinucleotide-based in vitro sequencing workflows (e.g. by flow cell-based sequencing, whereby template nucleic acid molecules are sequenced in vitro. In vitro template nucleic acid molecules can include, for example, synthetically produced nucleic acids (such as by phosphoramidite chemistry), and other isolated nucleic acid molecules such as those extracted from a biological sample. Nucleic acid extraction from cells or other biological samples may be performed using any of a variety of techniques known to those of skill in the art. For example, a typical DNA extraction procedure may comprise: (i) collection of a cell or tissue sample from which DNA is to be extracted, (ii) disruption of cell membranes (i.e., cell lysis) to release DNA and other cytoplasmic components, (iii) treatment of the lysed sample with a concentrated salt solution to precipitate proteins, lipids, and RNA, followed by centrifugation to separate out the precipitated proteins, lipids, and RNA, and (iv) purification of DNA from the supernatant (e.g., using spin columns or paramagnetic beads) to remove detergents, proteins, salts, or other reagents used during the cell membrane lysis step. Examples of methods for performing nucleic acid (e.g., DNA and RNA) extraction are described in, for example, Ali et al. (2017) “Current Nucleic Acid Extraction Methods and Their Implications to Point-of-Care Diagnostics”, BioMed Research International 2017:9306564, and Dairawan et al. (2020), “The Evolution of DNA Extraction Methods”, Am J Biomed Sci & Res 8(1):39-45, the entire contents of each of which are incorporated herein by reference.

A variety of suitable commercial nucleic acid extraction and purification kits are consistent with the disclosure herein. Examples include, but are not limited to, the QIAamp® kits (for isolation of genomic DNA from human samples) and DNAeasy kits (for isolation of genomic DNA from animal or plant samples) from Qiagen (Germantown, Md.), or the Maxwell® and ReliaPrep™ series of kits from Promega (Madison, Wis.).

### Sequencing Library Preparation

For dinucleotide-based flow cell sequencing approaches described herein, a sequencing library (including the template nucleic acid molecule(s)) may be prepared prior to sequencing. Sequence library preparation may be performed using any of a variety of techniques known to those of skill in the art. Library preparation typically comprises performing the steps of, e.g., end repair, tailing, and ligation of adapter sequences to template nucleic acid fragments. Extracted nucleic acid molecules (e.g., DNA molecule), or fragments thereof, that are typically used as the input for sequencing library preparation often have overhangs containing single-stranded DNA (ssDNA overhangs), breaks in the phosphodiester backbone that exist on just one strand (nicks), and/or ssDNA regions internal to the duplex molecule (ssDNA gaps). End repair reactions (using, e.g., a combination of 3′ exonuclease digestion to remove 3′ overhangs and a strand displacing polymerase reaction using dNTPs to fill nicks and gaps) are used to correct these defects in order to maximize the yield for capturing and sequencing the extracted DNA, and result in the generation of blunt-ended, double-stranded DNA (dsDNA) molecules.

Tailing (e.g., A tailing) is an enzymatic method (using, e.g., a Taq DNA polymerase) for adding a non-templated nucleotide (e.g., an A nucleotide) to the 3′ end of a blunt-ended, double-stranded DNA molecule that facilitates the ligation of the adapter sequences used for sequencing.

One or more adapter sequences may then be ligated to the ends of the end-repaired and tailed template nucleic acid molecules. The adapter sequences may comprise, for example, (i) capture sequences (e.g., the Illumina p5 and p7 adapter sequences) that allow the nucleic acid molecules of the library to bind to a flow cell surface comprising complementary capture probes, (ii) amplification primer binding sites for use in performing reverse transcription and/or for generating clonally-amplified clusters on a flow cell surface, (iii) sequencing primer binding sites (e.g., the Illumina Rd1 and Rd2 sequencing primer binding site sequences) used to initiate sequencing. In addition to amplification and/or sequencing primer binding sites, in some instances the adapters may comprise a barcode sequence, e.g., a sample identification barcode sequence (such as the Illumina Index 1 and Index 2 sample identifier sequences).

Examples of methods for performing sequencing library preparation are described in, for example, Head et al. (2014), “Library construction for next-generation sequencing: Overviews and challenges”, BioTechniques 56(1):61-77, and Hess et al. (2020), “Library preparation for next generation sequencing: A review of automation strategies”, Biotechnology Advances 41:107537, the entire contents of each of which are incorporated herein by reference.

In some embodiments, a method of dinucleotide-based flow cell sequencing as provided herein includes “cyclic array sequencing” of amplified template nucleic acid molecules. Cyclic array flow cell sequencing involves performing multiple cycles of an enzymatic reaction on an array of spatially separated oligonucleotide features (e.g., clonally-amplified colonies of template nucleic acid fragments tethered to a support surface, e.g., a flow cell surface). In some embodiments, the template nucleic acid is modified with known adapter sequence(s) comprising, e.g., amplification and/or sequencing primer binding sites, and then affixed to the support surface (e.g., the lumen surface(s) of a flow cell) in a random or patterned array by hybridization to surface-tethered complementary capture probes (complementary to adapter sequences) on the support surface, clonally amplified, and then probed using the aforementioned dinucleotide-based sequencing method as described herein. In some aspects the dinucleotide-based flow cell sequencing is a massively parallel sequencing reaction, whereby each reaction cycle of the dinucleotide-based sequencing method is used to query only one (the “interrogation nucleobase”) of the template nucleic acid fragment in each oligonucleotide feature, but thousands to billions of template nucleic acid molecules may be processed in parallel. Performing repeated cycles is then used to progressively identify the nucleic acid sequence based on patterns of signal detection or absence of a signal associated with binding of a dinucleotide molecule to the template, as detected over the course of multiple reaction cycles. In some aspects, signal detection is based on the use of labeled dinucleotide molecules (e.g., fluorescently labeled dinucleotide molecules) and imaging (e.g., fluorescence imaging) of the array.

In some instances, the disclosed methods for performing nucleic acid sequencing (e.g., in vitro and/or flow cell sequencing) may comprise performing one or more steps (e.g., 1, 2, 3, 4, 5, or more than 5) steps of nucleic acid amplification. Amplification reactions with respect to in situ based sequencing methods as described herein are discussed previously. In some instances, one or more steps of nucleic acid amplification may be performed as part of sequencing library preparation and/or following sequencing library preparation. In some instances, one or more steps of nucleic acid amplification (e.g., using a solid-phase amplification technique such as bridge amplification) may be performed after the template molecules of a sequencing library have been tethered to a support surface (e.g., a flow cell surface) to generate clonally-amplified colonies of the tethered template nucleic acid fragments.

In some instances, nucleic acid amplification may be performed to amplify all of the nucleic acid molecules extracted from a biological sample (e.g., using a random primer sequence). In some instances, nucleic acid amplification may be performed to amplify a selected subset of nucleic acid molecules extracted from a biological sample (e.g., using one or more primer sequences designed to hybridize to portions of the sequences for one or more target nucleic acid molecules of interest, or to sequences adjacent thereto).

In some instances, nucleic acid amplification may be performed to amplify an entire sequencing library (e.g., using a primer sequence that hybridizes to a common amplification primer binding site in the sequencing library adapters). In some instances, nucleic acid amplification may be performed to amplify selected portions of the sequencing library (e.g., using one or more primer sequences designed to hybridize to one or more amplification primer binding sites associated with one or more identifier sequences (or barcodes) included in the sequencing library adapters).

Nucleic acid amplification may be performed using any of a variety of nucleic acid amplification techniques known to those of sill in the art, including both thermal and/or isothermal nucleic acid amplification techniques. Examples of suitable thermal nucleic acid amplification techniques include, but are not limited to, polymerase chain reaction (PCR), multiplexed PCR, nested PCR, bridge PCR, reverse transcription PCR (RT-PCR). Examples of suitable isothermal nucleic acid amplification techniques include, but are not limited to, rolling circle amplification (RCA), nucleic acid sequence-based amplification (NASBA), loop-mediated isothermal amplification (LAMP), strand displacement amplification (SDA), helicase-dependent amplification (HDA), nicking enzyme amplification reaction (NEAR), and recombinase polymerase amplification (RPA). Examples of methods for performing nucleic acid amplification are described in, for example, Gill et al. (2008), “Nucleic Acid Isothermal Amplification Technologies—A Review”, Nucleosides, Nucleotides, and Nucleic Acids 27:224-243, Fakruddin et al. (2013), “Nucleic acid amplification: Alternative method of polymerase chain reaction”, J Pharm Bioallied Sci. 5(4):245-252, and U.S. Pat. No. 8,143,008, the entire contents of each of which are incorporated herein by reference.

### Compositions and Kits

In some aspects, provided herein are compositions comprising any of the primers, polymerases, dinucleotide molecules, and/or primary probes (e.g., circular probes or circularizable probes or probe sets) described herein. Also provided herein are kits for sequencing nucleic acid molecules, including kits for sequencing and analysis of target nucleic acids in a biological sample according to any of the methods described herein.

In some embodiments, provided herein is a kit comprising any of the pluralities of dinucleotide molecules described herein. In some embodiments, the kit further comprises any of the primers described herein (e.g., a priming strand comprising a 3′ reversibly terminated nucleotide or a primer lacking a 3′ reversible terminator). In some embodiments, the kit further comprises any of the polymerases described herein. In some embodiments, the kit further comprises any of the 3′ reversibly terminated nucleotide molecules described herein.

In some embodiments, provided herein is a kit for in situ dinucleotide-based sequencing comprising a plurality of dinucleotide molecules as described herein, and one or more further components for performing the in situ sequencing reaction. In some embodiments, the one or more further components include a polymerase, a primer, reversibly terminated dinucleotide molecules, a support for a tissue or cell sample, or any combination thereof. In some embodiments, the kit further comprises any of the circular probes and/or circularizable probes or probe sets disclosed herein. In some embodiments, the kit comprises a polymerase for performing rolling circle amplification.

In some embodiments, provided herein is a kit for flow cell-based dinucleotide-based sequencing comprising a plurality of dinucleotide molecules as described herein, and one or more further components for performing the flow cell sequencing reaction. In some embodiments, the one or more further components include a polymerase, a primer, reversibly terminated dinucleotide molecules, a flow cell, primers, adapters for sequencing library preparation, or any combination thereof.

The various components of the kit may be present in separate containers or certain compatible components may be pre-combined into a single container. In some embodiments, the kits further contain instructions for using the components of the kit to practice the provided methods. In some embodiments, the four sets of dinucleotide molecules (as described elsewhere) may be provided together in a single container, such as a tube. In some embodiments, each of the four sets of dinucleotide molecules is provided in four separate containers. In some embodiments, two of the four sets are provided together in a first container, and the other two of the four sets are provided in a second container.

In some aspects, provided herein is a kit for sequencing a template nucleic acid molecule, comprising: a plurality of dinucleotide molecules; a primer designed to hybridize to the template nucleic acid molecule, wherein the primer may comprise a 3′ reversibly terminated nucleotide; a 3′ reversibly terminated nucleotide molecule; and a polymerase. In some embodiments, the plurality of dinucleotide molecules may comprise any of the combinations of labeled and/or non-labeled dinucleotide molecules described elsewhere herein.

In some aspects, provided herein is a kit for sequencing a template nucleic acid molecule, comprising: a first plurality of dinucleotide molecules; a primer designed to hybridize to the template nucleic acid molecule, wherein the primer may comprise a 3′ reversibly terminated nucleotide; a 3′ reversibly terminated nucleotide; and a polymerase. In some embodiments, the kit comprises an additional plurality of dinucleotide molecules. In some embodiments, the first plurality of dinucleotide molecules and the additional plurality of dinucleotide molecules are selected from pairwise combinations of A, T, U, C, and G. In some aspects, provided herein is a kit for sequencing a template nucleic acid molecule, comprising: a first plurality of dinucleotide molecules; a primer designed to hybridize to the template nucleic acid molecule, where the primer may comprise a 3′ reversibly terminated nucleotide; a 3′ reversibly terminated nucleotide; and a polymerase. In some embodiments, the 3′ reversibly terminated nucleotide is a 3′-O-blocked reversibly terminated nucleotide. In some embodiments, the 3′-O-blocked reversibly terminated nucleotide is a 3′-O-azidomethyl deoxynucleotide triphosphate (3′-O-azidomethyl-dNTP), a 3′-O-allyl deoxynucleotide triphosphate (3′-O-allyl-dNTP), or a 3′-O-amino deoxynucleotide triphosphate (3′-O-NH₂-dNTP). In some embodiments, the 3′ reversibly terminated nucleotide is a 3′-unblocked reversibly terminated nucleotide.

In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some aspects, the kit can also comprise any of the reagents described herein, e.g., wash buffer and ligation buffer. In some embodiments, the kits contain reagents for detection and/or sequencing, such as barcode detection probes or detectable labels. In some embodiments, the kits optionally contain other components, for example nucleic acid primers,

### Systems

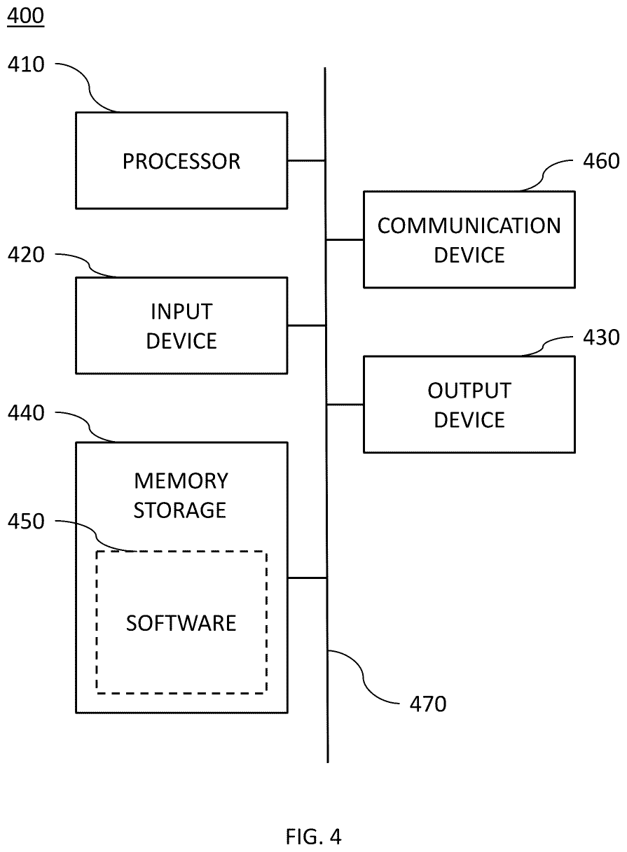

FIG. 4 illustrates an example of a computing device or system in accordance with one or more examples of the disclosure. Device 400 can be a host computer connected to a network. Device 400 can be a client computer or a server. As shown in FIG. 4, device 400 can be any suitable type of microprocessor-based device, such as a personal computer, workstation, server, or handheld computing device (portable electronic device), such as a phone or tablet. The device can include, for example, one or more of processor 410, input device 420, output device 430, memory/storage 440, and communication device 460. Input device 420 and output device 430 can generally correspond to those described above, and they can either be connectable or integrated with the computer.

Input device 420 can be any suitable device that provides input, such as a touch screen, keyboard or keypad, mouse, or voice-recognition device. Output device 430 can be any suitable device that provides output, such as a touch screen, haptics device, or speaker.

Storage 440 can be any suitable device that provides storage, such as an electrical, magnetic, or optical memory including a RAM, cache, hard drive, or removable storage disk. Communication device 460 can include any suitable device capable of transmitting and receiving signals over a network, such as a network interface chip or device. The components of the computer can be connected in any suitable manner, such as via a physical bus 470 or wirelessly.

Software 450, which can be stored in memory/storage 440 and executed by processor 410, can include, for example, the programming that embodies the functionality of the present disclosure (e.g., as embodied in the methods and systems described above). Software 450 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 440, that can contain or store programming for use by or in connection with an instruction execution system, apparatus, or device.

Software 450 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate, or transport programming for use by or in connection with an instruction execution system, apparatus, or device. The transport readable medium can include, but is not limited to, an electronic, magnetic, optical, electromagnetic, or infrared wired or wireless propagation medium.

Device 400 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 400 can implement any operating system suitable for operating on the network. Software 450 can be written in any suitable programming language, such as C, C++, Java, or Python. In various implementations, application software embodying the functionality of the present disclosure can be deployed in different configurations, such as in a client/server arrangement or through a web browser as a web-based application or web service, for example.

### Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide,” and “nucleic acid molecule,” used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

A “primer” as used herein, in some embodiments, is an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

In some instances, “ligation” refers to the formation of a covalent bond or linkage between the termini of two or more nucleic acids, e.g., oligonucleotides and/or polynucleotides, in a template-driven reaction. The nature of the bond or linkage may vary widely and the ligation, in some embodiments, is carried out enzymatically or chemically. As used herein, ligations are usually carried out enzymatically to form a phosphodiester linkage between a 5′ carbon terminal nucleotide of one oligonucleotide with a 3′ carbon of another nucleotide.

The term “about” as used herein refers to the usual error range for the respective value readily known to the skilled person in this technical field. Reference to “about” a value or parameter herein comprises (and describes) embodiments that are directed to that value or parameter per se.

As used herein, the singular forms “a,” “an,” and “the” comprise plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### EXAMPLES

The following examples are included for illustrative purposes only and are not intended to limit the scope of the present disclosure.

### Example 1: In Situ Dinucleotide Stochastic Sequencing

This example provides an example of a workflow for using dinucleotide stochastic sequencing to sequence a template nucleic acid molecule (e.g., DNA or RNA) in a tissue section. Use of fluorescently labeled dinucleotide molecules to stabilize a complex comprising the priming strand, template nucleic acid molecule, polymerase and a non-incorporated dinucleotide molecule may provide certain advantages such as stable retention of a fluorescent read-out, reduced “molecular scarring”, and/or longer sequence reads.

A tissue sample is obtained and cryo-sectioned onto a glass slide for processing. The tissue is fixed by incubating in 3.7% paraformaldehyde (PFA). To prepare for probe hybridization, a wash buffer is added to the tissue sample. The washed tissue sample is then contacted with a circularizable probe comprising a target sequence hybridization domain and a barcode sequence. The barcode sequence identifies a target analyte within the tissue sample. The circularizable probe is allowed to hybridize to the target analyte. The tissue sample is then contacted with a ligation reaction mix including ligase, and the circularizable probe is ligated to form a circular template for rolling circle amplification (RCA). The tissue sample is then incubated with an RCA mixture containing a Phi29 DNA polymerase and dNTP for RCA of the circularized probes. From this amplification, the RCA product (e.g., RCP) comprises the target sequence and a barcode sequence.

Next, the tissue sample is washed, and a sequencing process as illustrated in FIG. 5 is performed. First, the tissue sample is contacted with a primer. The primer is allowed to hybridize to a template strand comprising a sequence of the RCP (step 1 in FIG. 5, where the upper strand is the primer and the lower strand is the template nucleic acid molecule), and an extension reaction is performed to add a 3′ reversibly terminated nucleotide to the priming strand (step 2 in FIG. 5). The tissue sample is washed and then contacted with a polymerase and a first plurality of dinucleotide molecules to form a complex with a dinucleotide molecule, the polymerase, and the primer hybridized to the template strand, such that the dinucleotide molecule of the complex is complementary to the first two nucleotides downstream of the primer-bound portion of the template (step 3 in FIG. 5). The first plurality of dinucleotide molecules in this example includes four sets of dinucleotide molecules, each set including four different sequences sharing a same first (5′) nucleotide across the set and having a same label that is unique to the set. In this example, molecules of the first set are labeled with a green fluorophore (having an emission peak between 450 and 500 nm), molecules of the second set are labeled with a yellow fluorophore (having an emission peak between 500 and 550 nm), molecules of the third set are labeled with a red fluorophore (having an emission peal between 550 and 650 nm), and molecules of the fourth set are “labeled” with an absence of a fluorophore (e.g., not labeled with any fluorophore). The sixteen sequences are illustrated in the bottom left inset to FIG. 5. The complementarity between the two nucleotide residues of the non-incorporated dinucleotide molecule in the complex with the corresponding nucleotides of the template sequence helps stabilize the complex.

Next, fluorescence imaging is used to detect a signal associated with the presence of the dinucleotide molecule in the complex and thereby infer the identity of the nucleotide in the template nucleic acid molecule corresponding to the 5′ nucleotide of the dinucleotide molecule. Images for each of a plurality of detection channels configured to detect signals arising from labels (e.g., fluorescent dyes) conjugated to dinucleotide molecules present in the complex are acquired in each cycle of a multi-cycle sequencing run.

The tissue sample is next washed to disrupt the complex and remove the polymerase and complementary dinucleotide molecule (step 4 in FIG. 5). The primer bound to the template nucleic acid molecule is deprotected (step 5 in FIG. 5). A primer extension reaction is performed to incorporate a 3′ reversibly terminated nucleotide into the priming strand that is complementary to the identified nucleotide in the template nucleic acid sequence (step 6 in FIG. 5). Following primer extension, the sample is contacted with an additional plurality of dinucleotide molecules (also including the sixteen sequences shown in the bottom left inset to FIG. 5) and polymerase, and the entire cycle as described above is repeated (step 7 in FIG. 5) until the barcode of the RCP is sequenced.

### Example 2: Dinucleotide Stochastic Sequencing in a Flow Cell

This example provides a workflow for using dinucleotide stochastic sequencing to sequence a template nucleic acid molecule (e.g., DNA or RNA) immobilized on a flow cell surface. Use of fluorescently labeled dinucleotide molecules stabilize a complex comprising the priming strand, template nucleic acid molecule, and a non-incorporated dinucleotide molecule may provide certain advantages such as stable retention of a fluorescent read-out, reduced “molecular scarring”, and/or longer sequence reads.

RNA molecules extracted from a biological sample are processed as described elsewhere herein to create a sequencing library including template nucleic acid molecules to be sequenced. The template nucleic acid molecules in the library are attached to the surface of a flow cell at discrete locations (e.g., where each location comprises multiple clonally amplified copies of a single template nucleic acid molecule created using bridge amplification or a similar technique). Next, the sequencing process as illustrated schematically in FIG. 5 is performed. A primer that does not comprise a 3′ reversibly terminated nucleotide is introduced to the flow cell and allowed to hybridize to an immobilized template nucleic acid molecule (step 1 in FIG. 5). An extension reaction is then performed to add a 3′ reversibly terminated nucleotide to the priming strand (step 2 in FIG. 5). The immobilized template nucleic acid molecule is then contacted (in one or more steps) with a polymerase and a first plurality of dinucleotide molecules to form a complex of: i) a dinucleotide molecule that is complementary to the first two nucleotides downstream of the primer-bound portion of the template, ii) the polymerase, and iii) the primer hybridized to the template nucleic acid molecule (step 3 in FIG. 5). The first plurality of dinucleotide molecules in this example includes four sets of dinucleotide molecules, each set having four different sequences all including a same first (5′) nucleotide and labeled with a same fluorophore (or no fluorophore) (as illustrated in the bottom left inset to FIG. 5). The complementarity between the two nucleotide residues of the non-incorporated dinucleotide molecule in the complex with the corresponding nucleotides of the template sequence helps stabilize the complex.

Next, fluorescence imaging is used to detect a signal associated with the presence of the dinucleotide molecule in the complex and thereby infer the identity of the corresponding nucleotide in the template nucleic acid molecule. Images for each of a plurality of detection channels configured to detect signals arising from labels (e.g., fluorescent dyes) conjugated to dinucleotide molecules present in the transient complex are acquired in each cycle of a multi-cycle sequencing run.

The flow cell is then rinsed to disrupt the complex and remove the polymerase and complementary dinucleotide molecule (step 4 in FIG. 5). The primer bound to the template nucleic acid molecule is deprotected (step 5 in FIG. 5). A primer extension reaction is performed to incorporate a 3′ reversibly terminated nucleotide into the priming strand that is complementary to the identified nucleotide in the template nucleic acid sequence (step 6 in FIG. 5). Following primer extension, the sample is contacted with a second plurality of dinucleotide molecules (the second plurality also including the sixteen sequences shown in the bottom left inset to FIG. 5) and polymerase, and the entire cycle as described above is repeated (step 7 in FIG. 5) until the template nucleic acid molecule is sequenced.

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

1. A method for sequencing a template nucleic acid molecule comprising:
a) contacting a priming strand bound to the template nucleic acid molecule with a polymerase and a first plurality of dinucleotide molecules to form a complex comprising a 3′ terminus of the priming strand, the template nucleic acid molecule, the polymerase, and a first dinucleotide molecule of the first plurality of dinucleotide molecules, wherein the priming strand comprises a reversibly terminated nucleotide at its 3′ end such that the first dinucleotide molecule of the complex is not incorporated into the priming strand; and
b) detecting a presence of the first dinucleotide molecule in the complex.

2. (canceled)

3. The method of claim 1, further comprising:
c) deprotecting the reversibly terminated nucleotide at the 3′ end of the priming strand; and
d) performing an extension reaction to incorporate a reversibly terminated nucleotide molecule into an extended priming strand.

4. The method of claim 3, further comprising repeating steps (a)-(d) for at least one additional cycle using at least one additional plurality of dinucleotide molecules to detect a presence of a dinucleotide molecule in a complex and identify at least one additional complementary nucleotide in the template nucleic acid molecule.

5. The method of claim 1, wherein the first plurality of dinucleotide molecules comprises at least one of four sets of dinucleotide molecules, wherein each of the four sets comprises dinucleotide sequences comprising:
a same 5′ nucleotide that is unique for the set and one or more different 3′ nucleotides, wherein the one or more different 3′ nucleotides across the set are capable of base pairing with A, C, and G, and at least one of T and U.

6. The method of claim 1, wherein the first plurality of dinucleotide molecules comprises at least one of four sets of dinucleotide molecules, wherein each of the four sets comprises four different dinucleotide sequences comprising:
a same 5′ nucleotide that is unique for the set and four different 3′ nucleotides, wherein each dinucleotide molecule of the set is associated with a same detectable signal.

7. The method of claim 1, wherein the first plurality of dinucleotide molecules comprises at least one of four sets of dinucleotide molecules, wherein each of the four sets comprises dinucleotide sequences comprising:
one or more different 5′ nucleotides and a same 3′ nucleotide that is unique for the set, wherein the one or more different 5′ nucleotides across the set are capable of base pairing with A, C, and G, and at least one T and U.

8-14. (canceled)

15. The method of claim 5, wherein, for at least one of the four sets, a detectable signal is associated with a detectable moiety coupled to a corresponding dinucleotide molecule.

16. The method of claim 15, wherein, for at least three of the four sets, the detectable signal is associated with a detectable moiety coupled to a corresponding dinucleotide molecule.

17. The method of claim 15, wherein, for at least one of the four sets, the detectable moiety is coupled to a 3′ nucleotide of a corresponding dinucleotide molecule.

18. (canceled)

19. The method of claim 15, wherein the detectable moiety comprises a fluorophore.

20-24. (canceled)

25. The method of claim 4, wherein the at least one additional cycle comprises at least 2, 3, 4, 5, 10, 20, 30, 40, or 50 additional cycles.

26-32. (canceled)

33. The method of claim 4, wherein the first plurality of dinucleotide molecules and the at least one additional plurality of dinucleotide molecules each comprise sets of dinucleotide molecules that do not comprise a 3′ reversible terminator moiety.

34-38. (canceled)

39. The method of claim 1, wherein the template nucleic acid molecule comprises a DNA molecule.

40. (canceled)

41. The method of claim 1, wherein the template nucleic acid molecule comprises a target analyte nucleic acid molecule.

42. The method of claim 1, wherein the template nucleic acid molecule comprises a barcode sequence associated with a target analyte.

43-45. (canceled)

46. The method of claim 1, wherein the template nucleic acid molecule to be sequenced is attached to a solid support.

47. The method of claim 46, wherein the solid support comprises a sequencing flow cell.

48. The method of claim 1, wherein the template nucleic acid molecule is sequenced in situ in a cell sample or tissue sample.

49-67. (canceled)

68. The method of claim 1, further comprising identifying a complementary nucleotide in the template nucleic acid molecule based on the presence of the dinucleotide molecule in the complex.

69. A method for sequencing a template nucleic acid molecule comprising:
providing:
i) a priming strand bound to the template nucleic acid molecule, wherein the priming strand comprises a reversibly terminated nucleotide at its 3′ end; and
ii) one or more reagents comprising a polymerase molecule and a first plurality of dinucleotide molecules;
a) contacting the priming strand bound to the template nucleic acid molecule with the one or more reagents to form a complex comprising a 3′ terminus of the priming strand, the template nucleic acid molecule, the polymerase, and a first dinucleotide molecule of the first plurality of dinucleotide molecules, wherein the first dinucleotide molecule is blocked from being incorporated into the priming strand; and
b) detecting a presence of the first dinucleotide molecule in the complex.

70. The method of claim 69, further comprising identifying a complementary nucleotide in the template nucleic acid molecule based on the presence of the dinucleotide molecule in the complex.

71. The method of claim 69, wherein the first plurality of dinucleotide molecules comprises a set of four different dinucleotide sequences comprising:
a same 5′ nucleotide for each molecule of the set and four different 3′ nucleotides; or
four different 5′ nucleotides and a same 3′ nucleotide for each molecule of the set, wherein each dinucleotide molecule of the set is associated with a same detectable signal.
